(19) United States
(12) Patent Application Publication
(10) Pub. No.: US 2025/0135231 A1
(43) Pub. Date: May 1, 2025

(54) SYSTEMS AND METHODS FOR OPTIMIZATION OF RADIATION TREATMENT PLANNING TO IMPROVE IMMUNE RESPONSE

(71) Applicant: UNIVERSITY OF VIRGINIA PATENT FOUNDATION, CHARLOTTESVILLE, VA (US)

(72) Inventors: Krishni Wijesooriya, Charlottesville, VA (US); Cam Nguyen, Charlottesville, VA (US)

(21) Appl. No.: 18/935,373

(22) Filed: Nov. 1, 2024

Related U.S. Application Data
(60) Provisional application No. 63/595,288, filed on Nov. 1, 2023.
(60) Provisional application No. 63/715,464, filed on Nov. 1, 2024.

(51) Int. Cl. A61N 5/10; G16H 10/60; G16H 15/00; G16H 20/40; G16H 30/20; G16H 50/50

(52) U.S. Cl. CPC A61N 5/1038; A61N 5/1039; G16H 10/60; G16H 15/00; G16H 20/40; G16H 30/20; G16H 50/50

(57) ABSTRACT

Systems and methods are provided for estimating immune response effected by patient specific and plan specific radiation treatments (such as, e.g., SBRT). The systems and methods may take into account radiation impact on circulating immune blood cell types or sub-types, such as T lymphocytes, B lymphocytes, natural killer cells, erythrocytes, and/or neutrophils, and predict time dependent fractional blood count and cell kill following radiation therapy treatment. Additionally, the system, method, and computer readable medium provide parameters such as a dose dependent lymphocyte kill function and average net release rate of new lymphocytes into circulating blood (including promotion of cytotoxic T cells and suppression of lymphocytes in blood), which may be used for optimization of RT treatment plans.

### CROSS REFERENCE TO RELATED APPLICATIONS

The present application is based on, claims priority to, and incorporates herein by reference in its entirety for all purposes, U.S. Provisional Application Ser. No. 63/595,288, filed Nov. 1, 2023, and 63/715,464, filed Nov. 1, 2024.

### STATEMENT OF GOVERNMENT SUPPORT

This invention was made with government support under NIH-1R01CA234281 awarded by National Cancer Institute. The government has certain rights in the invention.

### BACKGROUND

Ionizing radiation is generally used as a powerful tool to kill cancer cells at target sites by DNA damage (hard kill). Radiation Therapy (RT) is generally known to give rise to two different immunological effects: 1) RT usually cases Radiation Induces Immune Suppression (RIIS) and lymphopenia, which are generally deleterious to the patient. For example, these side effects of RT may result in lower tumor control and/or tumor survival, and increase infections and hospitalizations. Lymphocytes, 80% of which are T cells, are highly radiosensitive and RIIS means destroying existing as well as newly created CTLs. 2) RT can sometimes cause a relatively rare effect known as an abscopal effect. While this effect is not well understood, and observations of it are sporadic, it has been postulated by the inventors that RT sometimes causes tumor cells to die in a way that releases tumor antigens, which may generate tumor-reactive effector T cells (modulate the immune system) that have the ability to specifically destroy cancer cells (soft kill). These may include Cytotoxic T Lymphocytes (CTLs) that recognize neo antigens specific to a patient's tumor. However, to date observations of potential immunomodulatory effects of localized RT on tumors have been sporadic and thus not sufficient to draw conclusions regarding how this effect is triggered or could be leveraged.

Current RT protocols do not optimize treatment planning to improve or leverage either of these two effects. For example, current RT protocols generally reduce focus on the tumor targets, rather than other factors like how the immune system will respond to the RT (in both potentially beneficial and deleterious ways). For example, they do not attempt to reduce or mitigate RIIS or to attempt to improve or facilitate generation of any positive immune response such as anti-tumor T cells. Furthermore, the inventors have determined that dose to different organs affect the immune suppression in different ways. As one example, even some typical parameters of current RT planning (such as maximum dose or mean dose for a radiation plan) are not necessarily the best parameters for evaluating the level of RIIS. Therefore, it is simply not possible with current approaches to determine even basic factors like lymph kill level of a plan by using the optimization parameters currently available in treatment planning systems.

Thus, it would be desirable to have systems and methods that carefully take into account multiple factors involved in immune response to RT (such as dynamics between the time dependent dose to structures and velocities of blood through those organs) to predict the effects that a given RT plan may have on suppressing immune response (e.g., expected lymphocyte loss, also called lymphocyte kill, lymph kill, or cell kill) as well as potential positive activation of beneficial immune response (e.g., generating tumor-reactive immune cells).

Therefore, there is need to optimize RT planning that systematically address both the generation of tumor-reactive effector T cells and RIIS.

### SUMMARY

The present disclosure provides systems, methods, and non-transitory computer readable storage medium that overcome the aforementioned drawbacks by using machine learning to the predicted immune modulatory effect of radio therapy (RT) on an abscopal effect, and RIIS.

The present disclosure is the first use of an algorithm for predicting post-treatment, time-dependent blood cell counts and lymphocyte sub type counts for RT treatments that include both RIIS and clonal expansion in a time dependent fashion. In addition to predictive capacity for individual patients, this algorithm can provide important limits of dosimetric and biological parameters that lead to the onset and abundance of clonal expansion in immune cells. The results were compared to measurements to quantify the predictability of the algorithm. This predictive algorithm will enable treatment plan design and optimization to give the lowest possible RIIS, and highest possible clonal expansion, while maintaining all other current clinical dosimetric requirements.

In one aspect of the present disclosure, a method is provided for generating a treatment plan based on predicting an immune effect induced by ionizing radiation in a subject. The method may comprise: assembling input data, wherein the input data includes imaging data of a subject, information relating to blood circulation of the subject, information relating to a radiation-based cancer therapy of the subject, and information concerning desirability of the immune effect; delivering the input data to a system for predicting immune response following radiation therapy (RT), wherein the system models immune cell toxicity in circulating blood caused by the RT; receiving an output from the system, comprising a predicted immune effect; determining a treatment plan for the subject using the output; and generating a report including at least one of the output or treatment plan.

In another aspect of the present disclosure, a system is provided for predicting an immune modulation effect induced by ionizing radiation in a cancer patient, comprising a user interface, a processor, and a memory. The memory stores an immune response predictor and software which, when executed by the processor, enables the system to perform a series of steps for optimizing radiation therapy (RT) treatment planning. The system receives imaging data of a target region in the cancer patient, processes this data to develop a spatial model of the region, and further updates the model to incorporate lymphatic and blood flow dynamics unique to the patient. The system identifies specific structures within the spatial model that require targeted radiation (target structures) and those needing minimized exposure (avoidance structures). Using this updated spatial model, the system generates a set of potential RT treatment plans and evaluates each plan with the immune response predictor to determine anticipated immune modulation effects. Based on these evaluations, the system selects one or more optimal treatment plans that minimize immune suppression while achieving therapeutic goals. And, the system outputs the selected optimal plans to the user, accompanied by information detailing the predicted immune responses associated with each plan, thereby facilitating informed treatment planning for enhanced patient outcomes.

In another aspect of the present disclosure, a non-transitory computer readable storage medium having software instructions stored thereon is described. The instructions, when executed by a computer processor, cause the computer to carry out steps comprising: delivering input data to a system for predicting the immune modulation effect of at least one of radiation induced immune suppression (RIIS) or generation of anti-tumor T cells following radiation therapy (RT), receiving an output from the system of the predicted immune modulation effect, determining a treatment plan for the subject using the output, and generating a report including at least one of the output or treatment plan.

These aspects are nonlimiting. Other aspects and features of the systems and methods described herein will be provided below.

### BRIEF DESCRIPTION OF THE DRAWINGS

The foregoing features of embodiments will be more readily understood by reference to the following detailed description, taken with reference to the accompanying drawings, in which:

FIG. 1 is a schematic diagram of one non-limiting example of a framework for implementing a model in accordance with the present disclosure.

FIG. 2 is a block diagram of an example immune modulation effect prediction system.

FIG. 3 is a block diagram of example components that can implement the immune modulation effect prediction system of FIG. 2.

FIG. 4 is a flow chart setting forth some non-limiting example steps of a process that can be implemented using a platform, for example, such as described with respect to FIG. 1.

FIG. 5 is a flow chart setting forth some non-limiting example steps of a process that can be implemented using a platform, for example, such as described with respect to FIG. 1

FIG. 6A is a schematic of the blood flow in the thorax and through the blood rich organs (heart and great vessels) and the transfer between the heart and the lungs that is used in the model.

FIG. 6B is a set of computed tomography (CT) images of an actual lung SBRT patient simulated with all organs to show the inclusion of all organs in the thorax.

FIG. 6C is a flow chart showing the workings of the predictive algorithm in increments of space (dx), and increments of time (dt).

FIG. 6D is a schematic of the blood flow through an organ at different time steps, where the irradiated blood at the previous time step has been moved up.

FIG. 7 is a plot of the time-dependent form of cell kill term K(t), recovery term R(t), and final prediction N(t).

FIG. 8A is a series of plots showing the average cumulative blood dose histogram for all patients after one fraction (top, bin width=0.02 Gy) and after all fractions (center, bin width=0.1 Gy), as well as the cumulative blood dose volume histograms and the corresponding cumulative cell kill contributions for two sample largest and smallest PTV volumes and for all patients (bottom).

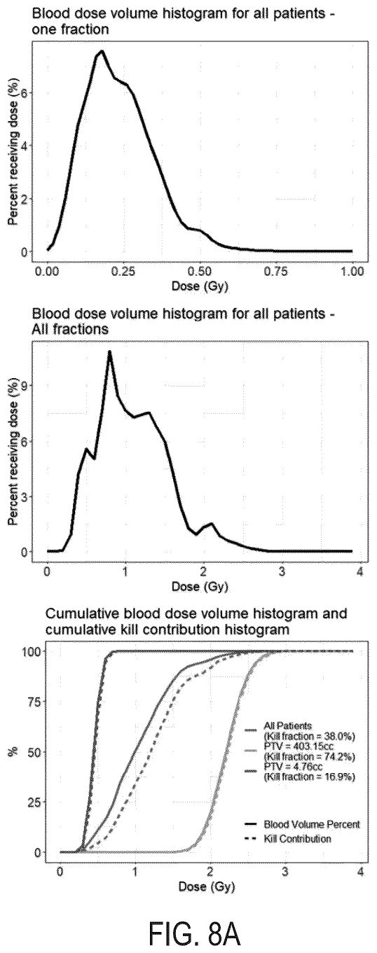

FIG. 8B is a series of plots showing the cumulative blood dose histogram for sample patients with different PTV volumes (top), different tumor locations (center) and different treatment delivery times (bottom).

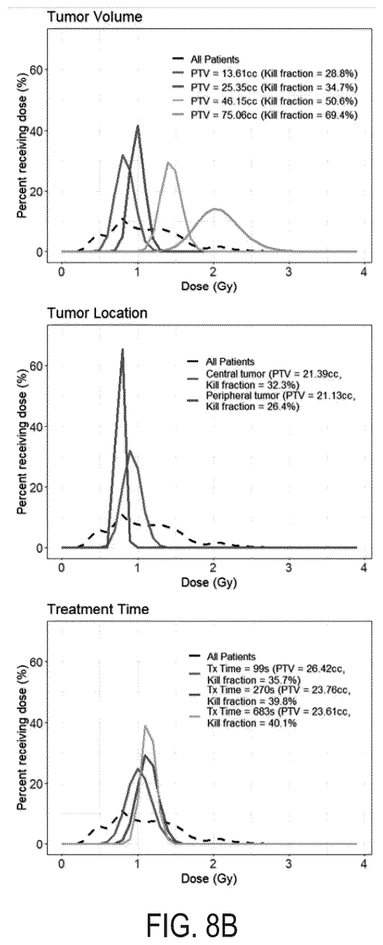

FIG. 9 is a histogram of the ALC difference (prediction-measurement) of all patients and which fold the prediction is on from the 10 folds.

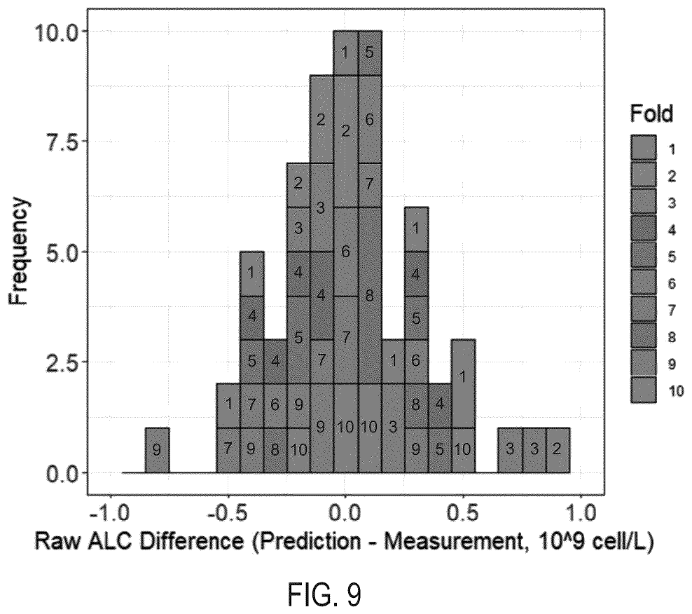

FIG. 10 is a pair of plots of the predicted post-treatment ALC count as a function of measured ALC count (left) and the cumulative distribution function (CDF) showing the proportion of patients with a given prediction accuracy (right).

FIG. 11A is a pair of plots showing the post-treatment ALC as a function of some key plan characteristics for both measurement and simulation. The Spearman's correlation coefficient r and p-value between the measured and predicted ALC and plan characteristic). The second plot shows the model accuracy—the absolute difference between prediction and measurement as a function of their variables for pre-treatment ALC (right).

FIG. 11B is a pair of plots showing the post-treatment ALC as a function of some key plan characteristics for both measurement and simulation. The Spearman's correlation coefficient r and p-value between the measured and predicted ALC and plan characteristic). The second plot shows the model accuracy—the absolute difference between prediction and measurement as a function of their variables for the minimum distance between PTV and heart (right).

FIG. 11C is a pair of plots showing the post-treatment ALC as a function of some key plan characteristics for both measurement and simulation. The Spearman's correlation coefficient r and p-value between the measured and predicted ALC and plan characteristic). The second plot shows the model accuracy—the absolute difference between prediction and measurement as a function of their variables for the days elapsed from treatment initiation to post ALC measurement (right).

FIG. 11D is a pair of plots showing the post-treatment ALC as a function of some key plan characteristics for both measurement and simulation. The Spearman's correlation coefficient r and p-value between the measured and predicted ALC and plan characteristic). The second plot shows the model accuracy—the absolute difference between prediction and measurement as a function of their variables for the PTV volume (right).

FIG. 11E is a pair of plots showing the post-treatment ALC as a function of some key plan characteristics for both measurement and simulation. The Spearman's correlation coefficient r and p-value between the measured and predicted ALC and plan characteristic). The second plot shows the model accuracy—the absolute difference between prediction and measurement as a function of their variables for the treatment delivery time (right).

### DETAILED DESCRIPTION

The following description provides a variety of examples in which techniques and innovations described herein can be implemented. In some examples, a predictive model may be leveraged, that allows the generation of patient-specific treatment plans that optimize a patient's likely immune response (whether to reduce RIIS or other immune suppression, activate beneficial immune response like stimulating T cell production in the body, or a combination thereof). In other examples, an RT regimen can be developed that creates an immune ‘hot’ environment increasing the efficacy of immunotherapy. Additionally, examples employing various treatment plan development and/or selection techniques described herein may reduce immune suppression due to radiation, which in turn can lead to fewer secondary infections, reduced rate of hospitalizations and medications, reduced health care costs, and/or increased survival outcomes.

The predictive algorithms utilized by the systems, methods, and non-transitory computer readable storage media described here are able to take into account radiation dose to circulating blood/lymphatics by coupling the time-dependence of the radiation delivery with a blood flow and lymphatics flow transport model that considers the transient time in the regional structures as well as the mixing of irradiated and non-irradiated blood/lymphatic volumes and migration between blood and lymphatics and lymphoid organs. The model for reducing RIIS and radiation treatment plan optimization tools significantly reduces the dose to circulating blood and lymphatics and minimize the expected immune cell loss for a given RT plan.

### Preliminary Considerations

The inventors have studied various effects of RT on immune response. While RIIS is fairly commonly observed, the beneficial effects of RT on immune response are more subtle and have not been directly studied in a controlled way. However, the systems and methods herein can nonetheless derive how beneficial effects can be promoted by deriving information from certain RT mechanisms.

Pre-clinical studies have shown that radiation facilitates dendritic cell maturation and migration and an increase in tumor-reactive T cells at ablative irradiation doses. At even higher doses, radiation has shown to increase the abundance of the immune suppressive cells such as regulatory T cells (Tregs) in the Tumor Micro Environment (TME) countering a desirable in-situ vaccine effect. Increasing the creation of anti-tumor immunity, new cytotoxic T cells (in particular CD4+ and CD8+ T cells which are specific to a particular patient's neo antigens) and control the creation of Tregs, while managing to reduce the RIIS, may retain this tumor specific adaptive immunity to fight against the tumor as well as distant metastasis. Additionally, certain immunotherapies seek to break the inhibitory mechanisms involved in immune reaction to the tumor, using Immune Checkpoint Blockade (ICB) therapies with anti-bodies such as anti-PD1, anti-PD_L1, and anti-CTLA4. Many of these immunotherapies rely on the presence of neo-antigen specific cytotoxic T cells in the body.

While an RT plan could be optimized to simply promote this adaptive immunity, the inventors posit that this should not be the sole goal; rather, RT is used in the first place to actually kill target tumors. So, any promotion of beneficial immune response should still be compatible with delivering a tumoricidal RT dose to the Planning Target Volume (PTV).

Additionally, it would be beneficial for systems and methods deploying techniques described herein to generate multiple-optimized RT plans that also are patient-specific (taking into account the patient's anatomy, tumor size/location, etc.) and feasible to accomplish using equipment on hand for most RT clinics. Thus, certain embodiments described herein may not only take into account a goal to kill tumor cells, a goal to reduce immune suppression, and a goal to promote beneficial immune response, but also patient- and clinic-specific factors.

Furthermore the way these goals and factors are used in the context of a given system or method described herein may include: prediction of immune response (e.g., overall suppression, activation of tumor-specific immune cells, increase in tumor antigen circulation, etc.) of a given RT plan; modification of an existing RT plan to improve one or more aspects of immune response; generation of a new RT plan to achieve desired immune outcomes; and/or generation of a set of RT plans with varying balances of goals relating to tumor eradication and immune response for review by physicians. Alternatively, embodiments could be used to assist in adjustment of RT plans during the course of the regimen. For example, at one or more time periods after a given RT dosing, a blood test or biopsy can be taken and immune response can be determined. If a desired goal of the RT plan is not being achieved (whether due to known or unknown factors), the RT plan can be modified via the techniques herein to more strongly emphasize or deemphasize the features of the plan correlating to that goal. (For example, if tumor cells are not being killed at the necessary or expected rate for a given patient/condition, a modification to an RT plan can be made that emphasizes dose strength, duration, or other factors for the target tumor, which may be at the expense of reducing RIIS or activation of immune response.)

One specific type of RT that can benefit from the techniques described herein is known as stereotactic body radiation therapy (SBRT). SBRT is an effective treatment for certain types of cancers, such as non-small cell lung cancer (NSCLC), that can achieve local control in more than 90% of cases. Beyond its primary mechanism for tumor control, SBRT has also been observed to sometimes seemingly modulate the immune system in ways which can improve outcomes. The inventors' experiments (described below) have leveraged the discovery that SBRT promotes generation of anti-tumor T-cells which may target distant metastases or residual disease not removed by the primary treatment. It can also stimulate tumor-infiltrating lymphocytes (TILs), leading to a mechanism known as “soft kill,” which can lead to more favorable outcomes. However, long-term outcomes for patients treated with SBRT remain poor, with 5-year survival rates of only 42% often driven by distant failures.

In addition to medical comorbidities, one potentially modifiable driver of poor long-term outcomes is radiation-induced immunosuppression (RIIS). RIIS stems from toxicity to highly radiosensitive lymphocytes and in severe cases can result in treatment-related lymphopenia (TRL), which has been associated with poor outcomes in numerous treatment sites. Reducing immune suppression is a promising avenue for improving patient outcomes following SBRT, especially with the increasing concurrent use of immunotherapy and radiation for cancer patients. Radiation treatments that reduce RIIS may allow more patients to be eligible for immunotherapy and may lead to improved survival following immunotherapy due to creating an immune hot environment. Furthermore, since the radiation induced immune suppression is a time-dependent function, understanding the time-dependent nature of RIIS may also improve outcomes by enabling optimal timing of immunotherapy administration for maximal tumor control.

As an example, the inventors' research leverages the discovery that specific treatment plan characteristics are linked to immunosuppression following RT for NSCLC. In advanced stage lung cancer, PTV volume, Lung V5-V10,10 mean thymus dose, and mean thoracic duct dose, V20 of thoracic spine, lung, and heart, blood rich and immune rich organs are linked to RIIS, while in early stage lung cancer thoracic spine V3, heart and lung dose volumes, and Heart+GV (integral dose, V5, V10, V15, V30), Thoracic spine (V1, V2, V5), lymph node stations (integral dose, V5, V10, V15) and rest of the body, external-PTV (integral dose, V1, V2, V5, V10, V15)) linked with near term RIIS, and acute long-term RIIS is correlated with only Heart+GV (V15, V20, V40). However, given the large heterogeneity in these threshold values, baseline immune status, tumor volumes, stage of the cancer and radiation treatment plans among patients, as well as the relative motion of the immune cells in the body with respect to the treatment beams, the inventors determined that a patient-specific approach would be more desirable.

Irradiation of circulating blood significantly reduces absolute lymphocyte counts (ALCs). Even a highly focused RT treatment such as SBRT can deliver a low but potentially toxic dose (>0.5 Gy) to circulating lymphocytes (CLs). Conventional RT planning and analysis tools, which aim to minimize the dose delivered to critical organs, are not equipped to reduce doses to a patient's blood pool. While these critical organs have well-defined contours and remain stationary during treatment, the blood pool is distributed throughout the body and circulates continuously. Individual blood cells can flow in and out of the irradiated field over the course of a treatment. To more accurately evaluate the dose delivered to CL for a given RT plan and estimate ALC, the inventors have determined that dynamics of blood flow as well as time-dependent dose delivery should be considered in the planning process.

Furthermore, in many cases, dose fractionations are 3-5 days and patients' immune levels vary rapidly after treatment, unlike standard fractionations where immune suppression is smoothly varying over time. Accurate predictions of time-dependent immune levels will help improve survival outcomes. For example, they may be valuable in deciding the optimum time window for administering immunotherapy. Additionally, a model that accounts for blood flow with organ-specific blood flow velocities in the thorax, as well as time-dependent lymphocyte replenishment, will be of great value due to its accuracy and flexibility in handling other individual organs.

### Systems and Methods

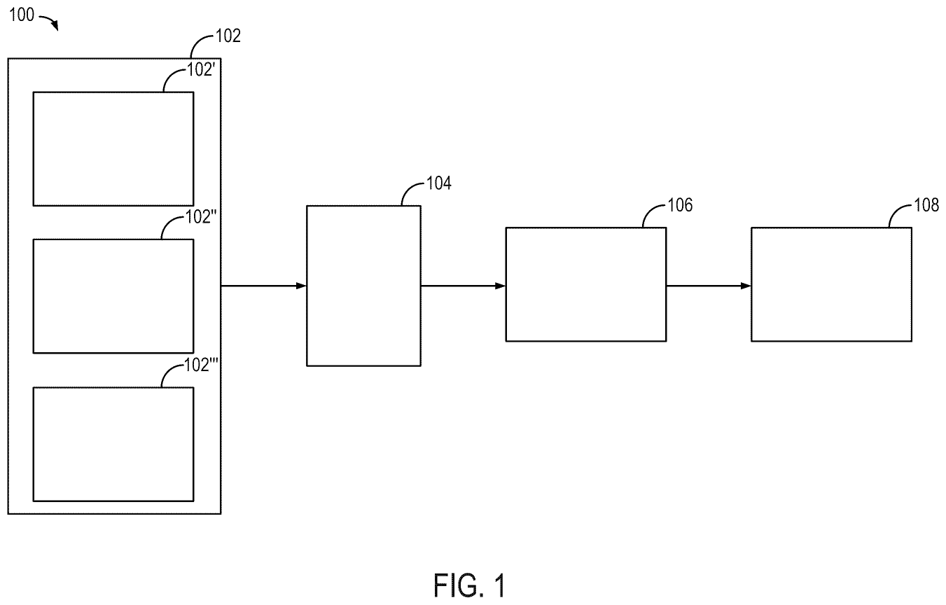

Referring to FIG. 1, an example of a system 100 for predicting an immune modulation effect induced by ionizing radiation in a subject is presented. The system 100 may be a processor configured to receive input data 102. In a non-limiting example, the input data 102 includes medical data of a subject 102′, radiation treatment plan data 102″, or other input data 102″. In a non-limiting example, the medical data 102′ may include imaging data. The imaging data may include, but is not limited to, computed tomography (CT) images or magnetic resonance (MR) images. Furthermore, the RT plan data 102″ may include RT structure sets, treatment plan parameters, dose maps, or a combination thereof. In a non-limiting example, the treatment plan parameters include at last one of a radiation dosage, duration, and frequency. The dose maps may be in Digital Imaging and Communications in Medicine (DICOM) format. In a non-limiting example, other input data 102″ may include a sample from a patient, such as, but not limited to, a blood sample indicating the immune cell count or tumor tissue biopsy.

The system 100 further includes a model for predicting the immune modulation effect 104 that receives the input data 102. In a non-limiting example, the immune modulation effect predicted by the model 104 is at least one of RIIS or the generation of anti-tumor T cells following RT. In one example, the model 104 is a linear-quadratic (LQ) model. The LQ model may model lymphocyte kill with the form K(Dᵢ)=1−e^{-(αD}^{ᵢ}^{+βD}^{ᵢ}^{²}⁾, where Dᵢ is an entry of a blood matrix representing a dose accumulated by an individual circulation lymphocyte after RT. In a non-limiting example, the LQ model models an interplay between (1) a time-dependent RT delivery, (2) a movement of blood, lymphatics, and lymphocytes, and (3) osmosis between primary organs, secondary organs, and non-lymphoid organs. The interplay may further include (4a) a blood cell kill, (4b) a bone marrow kill and a bone marrow recovery time, or (4c) a combination thereof. Further details of the model for predicting the immune modulation effect are provided in the Example below.

In a non-limiting example, the RIIS includes a change in a count of at least one blood cell type and at least one lymphocyte sub-population. For example, the at least one lymphocyte sub-population includes T cells, B cells, or natural killer (NK) T cells. Furthermore, the T cells may include CD3+. CD4+, CD8+, CD19+, or CD56+.

Briefly, in addition to their vital function in the body's general defenses against infections, lymphocyte sub types also play very important roles in tumor suppression: expressions of CD3+ and CD4+ sub types of lymphocytes were significantly associated with overall survival of NSCLC patients, CD8+ and CD56+ cells exert antitumor activity via antigen specific and antigen nonspecific mechanisms, elevated circulating CD19+ lymphocytes can predict survival in patients with gastric cancer. There have been many other studies which have shown that CD3+, CD4+, CD8+, CD19+, and CD56+ subsets are important in antitumor immunity, and Immune suppression may increase the risk of tumor growth and metastasis. By analyzing data from large cohorts of human tumors, it has been established that infiltration of the primary tumor by memory T cells, particularly of the T helper types and cytotoxic types (CD8+), is the best prognostic factor of disease-free survival and overall survival at all stages of clinical disease. High numbers of CD3+, CD8+ T cells in the primary tumor correlated with lack of metastasis and protection against tumor recurrence. Thus, it is beneficial to promote and/or preserve the existing tumor reactive T cells in the immune system, and thereby exert an immunogenic kill to the tumor.

Furthermore, the tumor microenvironment (TME) consists of innate immune cells: NK T cells, neutrophils, macrophages, mast cells, myeloid-derived suppressor cells (MDSCs), and Dendritic Cells (DC) as well as adaptive immune cells which are the T and B lymphocytes. T-cells fall into two broad categories, CD8+ (cytotoxic), and CD4+ (helper). When a cytotoxic CD8+ T cell encounters a tumor cell displaying the antigen it recognizes on MHC class I, it will kill tumor cell by the direct release of cytotoxic substances such as perforins, and granzymes. CD4+ helper T cells are a major source for TGF-β production and that TGF-β helps activation of nearby immune cells including CD8+ T cells, natural killer (NK) T cells, B cells, and innate immune cells. However, Tregs may counteract such anti-tumor activity by CD8+ T cells at the tumorsite, by exerting immune suppression in co-operation with myeloid-derived suppressor cells (MDSCs) and tumor-associated macrophages (TAMs).

When an antigen presenting cell (APC) such as the DC travel to the T cell zone of the lymph node carrying the neo antigen, it can interact with the naïve T cell that has the corresponding T cell receptor (TCR) that recognizes particular neo-antigen. However, T-cell activation requires two steps with distinct signaling processes: signal 1: engagement of the TCR and specific antigen presented by an MHC class I or II molecule, and signal 2: ligation of the co-stimulatory molecule CD28 on T cells with B7 on activated DC. Naïve T cell will now get activated and this leads to clonal expansion creating thousands of activated new T cells with the same TCR. This process can lead to an increase in CD4+ and CD8+ T cells post SBRT as observed in the previous trial. On the other hand, high radiation doses upregulates CTLA-on a subset of CD4+ leading to Tregs. Tregs dampen the immune system both at the lymph node and at the tumor site. Tregs are shown to be less radiation sensitive than the other T cells.

Referring back to FIG. 1. The system 100 further receives an output 106 from the model 104 of the predicted immune modulation effect. The output may include a score, index, or metric of RIIS, anti-tumor T cell generation, or a combination thereof. For example, the prediction output 106 includes post-treatment time dependent blood cell counts and lymphocyte sub type counts. In other embodiments, the output may include one or more suggested RT plans, modifications of RT plans, or other outputs as described herein.

In a non-limiting example, the system 100 further generates a report 108 including at least one of the output 106 or a treatment plan. For example, the treatment plan may be based on the output 106 to optimize radiation dose, delivery, time, delay between treatments, etc.

In a non-limiting example, the RT includes, but is not limited to, SBRT. The RT may target other anatomical areas of a subject that may be treated with RT.

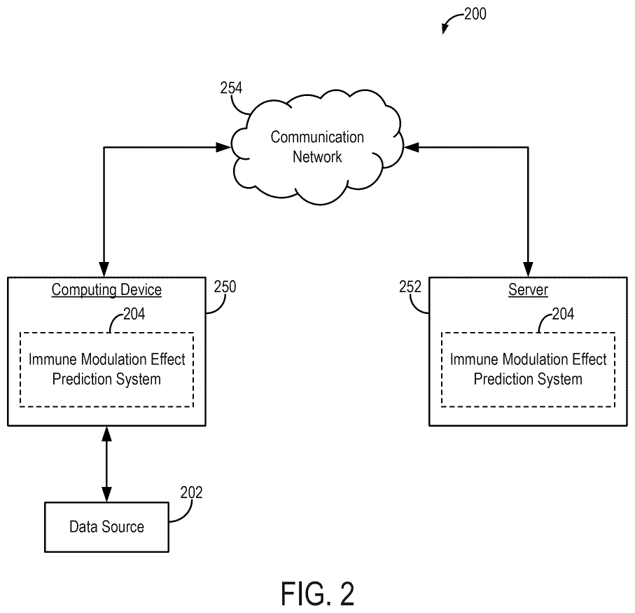

Referring now to FIG. 2, an example of a system 200 for predicting an immune modulation effect induced by ionizing radiation in a subject in accordance with some embodiments of the systems and methods described in the present disclosure is shown. As shown in FIG. 2, a computing device 250 can receive one or more types of input data (e.g., medical data of a patient, radiation treatment plan data, etc.) from data source 202. In some embodiments, computing device 250 can execute at least a portion of an immune modulation effect prediction system 204 to generate at least one of a prediction of RIIS or the generation of anti-tumor T cells, or a treatment plan from data received from the data source 202.

Additionally or alternatively, in some embodiments, the computing device 250 can communicate information about data received from the data source 202 to a server 252 over a communication network 254, which can execute at least a portion of the immune modulation effect prediction system 204. In such embodiments, the server 252 can return information to the computing device 250 (and/or any other suitable computing device) indicative of an output of the immune modulation effect prediction system 204.

In some embodiments, computing device 250 and/or server 252 can be any suitable computing device or combination of devices, such as a desktop computer, a laptop computer, a smartphone, a tablet computer, a wearable computer, a server computer, a virtual machine being executed by a physical computing device, and so on. The computing device 250 and/or server 252 can also reconstruct images from the data.

In some embodiments, data source 202 can be any suitable source of data, such as an imaging system or another computing device (e.g., a server storing measurement data, images reconstructed from measurement data, processed image data, radiation treatment plan data), and so on. In some embodiments, data source 202 can be local to computing device 250. For example, data source 202 can be incorporated with computing device 250 (e.g., computing device 250 can be configured as part of a device for measuring, recording, estimating, acquiring, or otherwise collecting or storing data). As another example, data source 202 can be connected to computing device 250 by a cable, a direct wireless link, and so on. Additionally or alternatively, in some embodiments, data source 202 can be located locally and/or remotely from computing device 250, and can communicate data to computing device 250 (and/or server 252) via a communication network (e.g., communication network 254).

In some embodiments, communication network 254 can be any suitable communication network or combination of communication networks. For example, communication network 254 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 254 can be a local area network, a wide area network, a public network (e.g., the Internet), a private or semi-private network (e.g., a corporate or university intranet), any other suitable type of network, or any suitable combination of networks. Communications links shown in FIG. 2 can each be any suitable communications link or combination of communications links, such as wired links, fiber optic links, Wi-Fi links, Bluetooth links, cellular links, and so on.

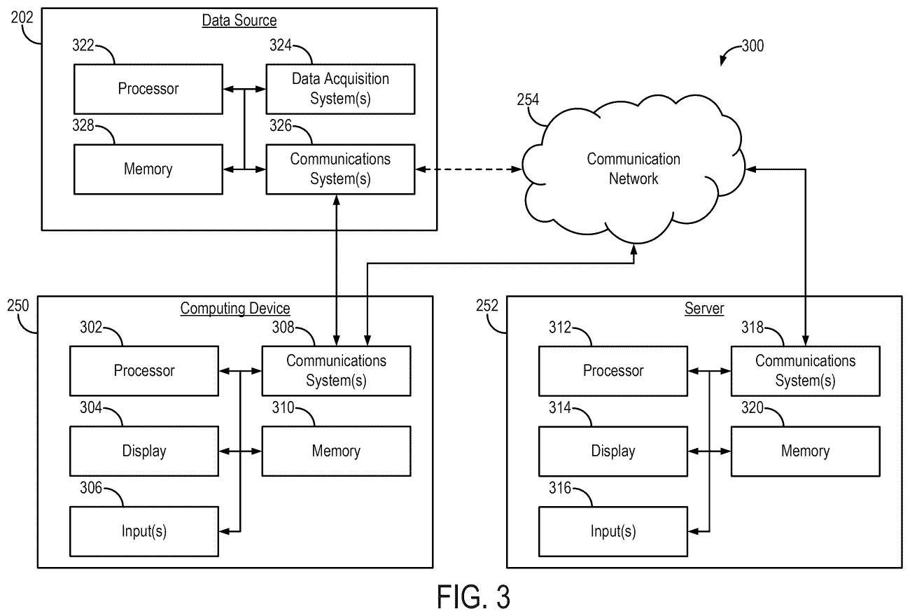

Referring now to FIG. 3, an example of hardware 300 that can be used to implement data source 202, computing device 250, and server 252 in accordance with some embodiments of the systems and methods described in the present disclosure is shown.

As shown in FIG. 3, in some embodiments, computing device 250 can include a processor 302, a display 304, one or more inputs 306, one or more communication systems 308, and/or memory 310. In some embodiments, processor 302 can be any suitable hardware processor or combination of processors, such as a central processing unit (“CPU”), a graphics processing unit (“GPU”), and so on. In some embodiments, display 304 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 306 can include any suitable input devices and/or sensors that can be used to receive user input, such as a keyboard, a mouse, a touchscreen, a microphone, and so on.

In some embodiments, communications systems 308 can include any suitable hardware, firmware, and/or software for communicating information over communication network 254 and/or any other suitable communication networks. For example, communications systems 308 can include one or more transceivers, one or more communication chips and/or chip sets, and so on. In a more particular example, communications systems 308 can include hardware, firmware, and/or software that can be used to establish a Wi-Fi connection, a Bluetooth connection, a cellular connection, an Ethernet connection, and so on.

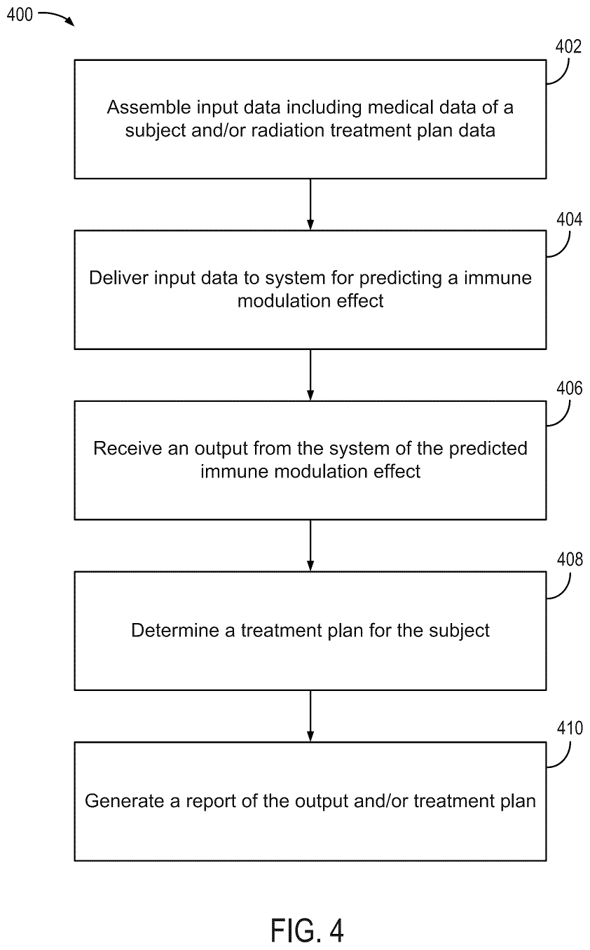

In some embodiments, memory 310 can include any suitable storage device or devices that can be used to store instructions, values, data, or the like, that can be used, for example, by processor 302 to present content using display 304, to communicate with server 252 via communications system(s) 308, and so on. Memory 310 can include any suitable volatile memory, non-volatile memory, storage, or any suitable combination thereof. For example, memory 310 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 310 can have encoded thereon, or otherwise stored therein, a computer program for controlling operation of computing device 250. In such embodiments, processor 302 can execute at least a portion of the computer program to present content (e.g., images, user interfaces, graphics, tables), receive content from server 252, transmit information to server 252, and so on. For example, the processor 302 and the memory 310 can be configured to perform the methods described herein (e.g., the methods of FIGS. 4A-4B).

In some embodiments, server 252 can include a processor 312, a display 314, one or more inputs 316, one or more communications systems 318, and/or memory 320. In some embodiments, processor 312 can be any suitable hardware processor or combination of processors, such as a CPU, a GPU, and so on. In some embodiments, display 314 can include any suitable display devices, such as an LCD screen, LED display, OLED display, electrophoretic display, a computer monitor, a touchscreen, a television, and so on. In some embodiments, inputs 316 can include any suitable input devices and/or sensors that can be used to receive user input, such as a keyboard, a mouse, a touchscreen, a microphone, and so on.

In some embodiments, communications systems 318 can include any suitable hardware, firmware, and/or software for communicating information over communication network 254 and/or any other suitable communication networks. For example, communications systems 318 can include one or more transceivers, one or more communication chips and/or chip sets, and so on. In a more particular example, communications systems 318 can include hardware, firmware, and/or software that can be used to establish a Wi-Fi connection, a Bluetooth connection, a cellular connection, an Ethernet connection, and so on.

In some embodiments, memory 320 can include any suitable storage device or devices that can be used to store instructions, values, data, or the like, that can be used, for example, by processor 312 to present content using display 314, to communicate with one or more computing devices 250, and so on. Memory 320 can include any suitable volatile memory, non-volatile memory, storage, or any suitable combination thereof. For example, memory 320 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 320 can have encoded thereon a server program for controlling operation of server 252. In such embodiments, processor 312 can execute at least a portion of the server program to transmit information and/or content (e.g., data, images, a user interface) to one or more computing devices 250, receive information and/or content from one or more computing devices 250, receive instructions from one or more devices (e.g., a personal computer, a laptop computer, a tablet computer, a smartphone), and so on.

In some embodiments, the server 252 is configured to perform the methods described in the present disclosure. For example, the processor 312 and memory 320 can be configured to perform the methods described herein (e.g., the method of FIG. 4).

In some embodiments, data source 202 can include a processor 322, one or more data acquisition systems 324, one or more communications systems 326, and/or memory 328. In some embodiments, processor 322 can be any suitable hardware processor or combination of processors, such as a CPU, a GPU, and so on. In some embodiments, the one or more data acquisition systems 324 are generally configured to acquire data, images, or both, and can include medical imaging systems. Additionally or alternatively, in some embodiments, the one or more data acquisition systems 324 can include any suitable hardware, firmware, and/or software for coupling to and/or controlling operations of the medical imaging systems. In some embodiments, one or more portions of the data acquisition system(s) 324 can be removable and/or replaceable.

Note that, although not shown, data source 202 can include any suitable inputs and/or outputs. For example, data source 202 can include input devices and/or sensors that can be used to receive user input, such as a keyboard, a mouse, a touchscreen, a microphone, a trackpad, a trackball, and so on. As another example, data source 202 can include any suitable display devices, such as an LCD screen, an LED display, an OLED display, an electrophoretic display, a computer monitor, a touchscreen, a television, etc., one or more speakers, and so on.

In some embodiments, communications systems 326 can include any suitable hardware, firmware, and/or software for communicating information to computing device 250 (and, in some embodiments, over communication network 254 and/or any other suitable communication networks). For example, communications systems 326 can include one or more transceivers, one or more communication chips and/or chip sets, and so on. In a more particular example, communications systems 326 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 328 can include any suitable storage device or devices that can be used to store instructions, values, data, or the like, that can be used, for example, by processor 322 to control the one or more data acquisition systems 324, and/or receive data from the one or more data acquisition systems 324; to generate images from data; present content (e.g., data, images, a user interface) using a display; communicate with one or more computing devices 250; and so on. Memory 328 can include any suitable volatile memory, non-volatile memory, storage, or any suitable combination thereof. For example, memory 328 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 328 can have encoded thereon, or otherwise stored therein, a program for controlling operation of data source 202. In such embodiments, processor 322 can execute at least a portion of the program to generate images, transmit information and/or content (e.g., data, images, a user interface) to one or more computing devices 250, receive information and/or content from one or more computing devices 250, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

Referring now to FIG. 4, a method 400 is provided for predicting an immune modulation effect induced by ionizing radiation in a subject. The method 400 may be implemented using system 100 of FIG. 1 as previously described. The method includes assembling input data in step 402. As used herein, “assembled” refers to, for example, that the data may be accessed from patient records, databases, or storage mediums, or may be acquired or derived. The input data, as previously described, may include at least one of medical data of a subject, radiation treatment plan data, or a combination thereof. At step 404, the input data may be delivered to a system for predicting an immune modulation effect. As described previously, the system may include a model for predicting an immune modulation effect. At step 406, an output of the predicted immune modulation effect is received. At step 408, the system may determine a treatment plan for the subject, as previously described with reference to FIG. 1. Furthermore, a report may be generated at step 410. As previously described, the repot may include at least one of the output or the treatment plan.

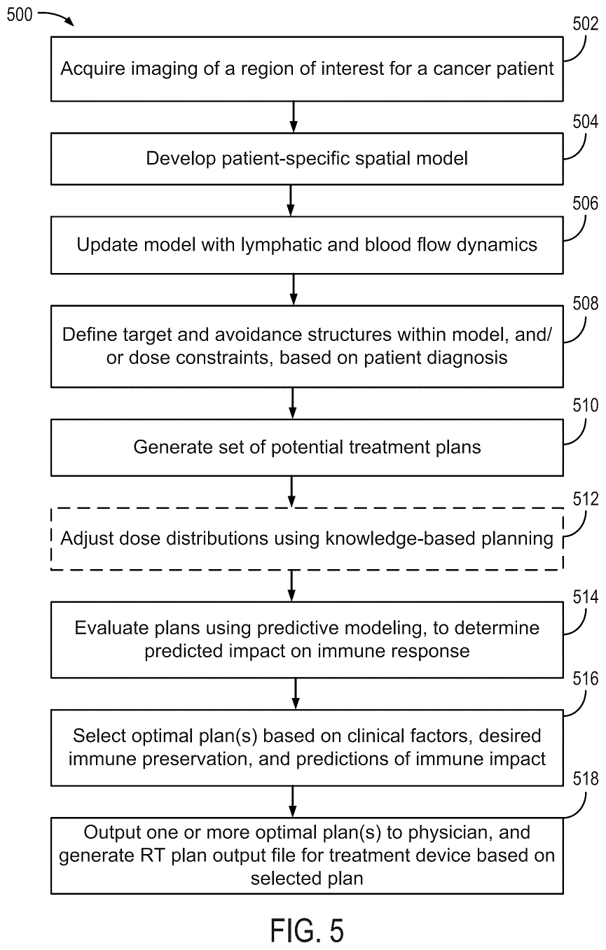

Referring to FIG. 5, a method 500 for generating RT plans is shown. The method 500 and/or a subset of its steps, may be combined with or used to support steps in the method of FIG. 4. Method 500 may also be embodied in computer software stored in a memory, to be run in a processor such as described above with respect to FIGS. 1-3.

At step 502, an image of a region of interest of a cancer patient is acquired (i.e., imaging is taken of the region of interest, image data is acquired from a database, etc.). In a non-limiting example, high-resolution images (e.g., CT, MRI) are gathered of the cancer patient's tumor(s) and surrounding anatomy to serve as data for patient-specific spatial modeling.

At step 504, a patient-specific spatial model may be developed whereby tumor(s) or other radiation targets, organs-at-risk (OARs), lymphatic structures, and blood-rich tissues can be delineated. In a non-limiting example, the model may be a three-dimensional (3D) model generated from two-dimensional (2D) imaging slices. In other examples, the model may be slice-based or based solely on 2D imaging. In some embodiments, the model may be displayed to a user to help visualize the relationship between the tumor/targets, as well as organs and immune-sensitive structures that may be desired to be protected from radiation to the extent possible under a given plan.

At step 506, the model may be updated with lymphatic and blood flow dynamics. In a non-limiting example, dynamic blood and lymphatic flow data are incorporated into the model, allowing the model to estimate the distribution and accumulation of radiation dose in circulating immune cells over time. This simulation helps predict immune suppression effects. In some embodiments, a spatial model may be updated to include flow and directional information relating to how and where blood and lymphatic cells originate and circulate within the region of interest.

In a non-limiting example, target and avoidance structures may be defined in the model at step 508. For example, specific structures may be identified in the model that require targeted radiation (e.g., tumor tissue) and those that need dose minimization (e.g., lymph nodes, blood-rich organs) to protect immune function. In some examples, certain organs that often are considered “organs at risk” or which otherwise would typically be ideally avoided in an RT plan may be auto-identified via a machine-learning classifier. For example, computer vision techniques may be utilized to segment and locate major structures in 2D images, 3D images, and/or a 3D model and/or a neural network may be utilized to identify structures corresponding to these typical organs (e.g, heart, spine, lungs, etc.). Similarly, various methods for auto-identification of tumors or lesions can also be employed. In other examples, a user interface may be presented to allow a physician (radiologist, oncologist) or other healthcare professional to label such structures by visual inspection and/or to confirm predicted labeling from a classification algorithm.

In some embodiments, 2, 3, 4, or more types of labels may be used, such as “RT target,” “OAR,” “lymphatic-generating tissue,” “major lymphatic-generating tissue,” “minor lymphatic-generating tissue,” and the like. In other embodiments, a user may be given the option to rate or weight the desirability of irradiation of each structure, from an RT target being highly desirable or necessary to receive a given radiation dose, to particularly sensitive OARs or lymphatic tissues being highly desirable not to receive radiation or not to receive more than a given dose. This labeling and/or weighting may be utilized in some examples to inform RT planning including dose constraints for treatment planning.

At step 510, a set of candidate treatment plans may be generated and optimized. In some embodiments, automated software-based treatment planners may be utilized to generate an initial set of candidate treatment plans, such as the Eclipse® package from Varian Medical Systems, the RayStation package from RaySearch Laboratories, and other similar planners. In other embodiments, physicians may input existing RT treatment plans or plans they developed to be patient-specific. For example, in some embodiments, the number of candidate plans may be 5, 10, 25, 30, 100, or similar values.

In a non-limiting example, multi-criteria optimization (MCO) may be utilized to create a series of candidate plans, or optimize/cull a set of existing candidate plans. In some examples, process 500 may employ an MCO-type approach by calculating multiple possible dose distributions, each optimized to meet competing criteria such as tumor control probability and normal tissue complication probability; in accordance with various techniques described herein, this may also be informed by user input regarding relative importance for a given patient of ensuring tumor cell death, limiting RIIS, promoting beneficial tumor antigen circulation, and/or promoting beneficial tumor-directed T cell activation, and similar goals. The MCO process may also be informed by labeling or weighting of given structures within the region of interest, as described above. Process 500 may iteratively refine potential dose distributions based on the defined dose constraints and other factors.

In addition to, or as an alternative to, an MCO process, a modified knowledge-based planning (KBP) process may also be utilized at step 512 to refine, optimize, and/or cull a set of candidate plans. In such example, a machine learning model may be trained on a database of RT plans that successfully achieved both tumor kill effect and desirable immune response. In some embodiments, imaging or spatial models associated with those RT plans may be labeled or weighted according to the process described above, to provide further information to the machine learning model. Thus, process 500 can compare the current patient's current patient's anatomy (based on spatial model), tumor locations, and desired outcomes to similar cases, and guide the generation, optimization or culling of candidate RT plans to align with optimal past outcomes.

At step 514, one or more optimal plans (e.g., plans resulting from the generation, optimization, and culling steps above) may be evaluated using predictive modeling. In a non-limiting example, the evaluation and selection may be performed automatically and/or to determine a predicted impact to the given patient's specific immune response. Evaluating the optimal plan may include analyzing each candidate plan's predicted impact on immune suppression (RIIS) using the predictive model. Some embodiments may perform the evaluation by utilizing the spatial/dynamic model generated for the specific region of interest for the specific patient, to assess each optimal plan's predicted impact on immune function (e.g., lymphocyte preservation, tumor-directed T-cell generation, etc.). The model simulates radiation dose delivery across dynamic blood and lymphatic flow to predict immune cell exposure. The result of such model may vary, including: a presentation to a user of predicted impacts for the optimal plans; adjustments to the one or more plans to further reduce radiation-induced immune suppression (RIIS) and/or promote an abscopal effect; a ranking of the one or more optimal plans based on how closely they would achieve the desired outcome and/or how likely they are to achieve the desired outcome; or simply the top 2, 3, 4, 5, 10, etc. most optimal plans.

In addition to (or alternative to) the model evaluation, another approach for identifying or culling optimal plans may include a dose-volume histogram analysis (DVH). DVHs provide a graphical representation of dose distribution across the tumor and surrounding tissues. In some embodiments, each candidate plan (or a set of near-optimal plans) can be analyzed through its DVH to check whether it meets the prescribed dose thresholds for tumor coverage and immune preservation.

At step 516, an optimal plan may be selected based on clinical factors, a desired immune modulation effect, desired immune preservation, and prediction of immune impact. In a non-limiting example, selecting the optimal plan includes choosing the plan that provides effective tumor control while minimizing immune suppression, guided by dose-volume histograms (DVHs) and lymphocyte preservation goals.

In some embodiments, process 500 may select the optimal plan (or a desired, small set of optimal plans) by comparing a number of factors of the candidate plans such as their DVHs, immune response predictions, and clinical feasibility. The optimal plan may be chosen as the one that best balances tumor eradication with immune preservation and/or coordination with immunotherapy, providing high precision and meeting the patient's unique anatomical and biological needs. Thus, as described above, a physician's or other user's input regarding desired goals, relative importance of goals, availability equipment, and patient-specific attributes (e.g., age, likely ability to tolerate radiation, aggressiveness of tumor growth/spreading, etc.), can be utilized to automatically choose the best RT plan or top set of RT plans. In other embodiments, a goodness of fit or alignment score can also be given to each of a set of optimal plans, as an amalgam of how closely a plan fits the desired goals, balance of goals, and constraints, as well as how likely the outcomes are based on model confidence.

At step 518, the one or more optimal plans may be output to a physician, and may include generating one or a set of RT output files for treatment devices based on selected plans, such as DICOM RT files and any associated imaging guidance plans.

In another aspect of the present disclosure, an existing RT plan may be modified mid-course to improve one or more aspects of immune response. For example, new input data may be collected and used to update the model. The new input data may include imaging data, as well as patient samples. In a non-limiting example, a patient's blood may be sampled to evaluate the change in immune cell count after RT. Depending on the type of change in immune cell count, one or more parameters of the RT may be updated accordingly. For example. The change in the immune cell count may indicate too much immune cell suppression, successful immune cell expansion, etc.

The examples below provide details related to the development and use of the systems and methods as described herein. It is noted that these examples were performed as part of illustrative experiments performed by the inventors, and are not intended to be limiting.

### EXAMPLES AND EXPERIMENTS

As described herein, an illustrative approach was taken to development and use of a model that predicts long-term acute RIIS for lung SBRT by estimating the dose delivered to circulating blood lymphocytes. The approach models dose within all organs in the thorax surrounding the treatment area up to a threshold of 40 cGy per fraction (it has been reported that 50 cGy is a threshold for lymphocyte kill) and accounts for patient-specific anatomy, treatment plan, and baseline immune status to predict time-dependent post-treatment ALC. The model described herein is configured to interface with treatment planning systems (based on DICOM format) and generate fast predictions to augment decision making during treatment planning.

The output of this illustrative approach can be utilized for the generation, optimization, evaluation, and selection of RT plans.

### 2.1 Dynamic Delivery Stimulation

An example model developed by the inventors included three conceptual components—(i) a simulation of dynamic dose delivery to blood flowing through key organs with random mixing of blood outside the radiation dose area, (ii) a dose-dependent cell kill model, and (iii) a time-dependent lymphocyte death and regeneration model. Blood flow dynamics were modeled using a Monte-Carlo style simulation in which local flow velocities were determined using published organ-specific cardiac outputs and organs' cross-sectional areas in patient-specific CT images. (Of course, in various other examples, the actual patient's cardiac outputs and cross-sectional areas could be used, rather than or in addition to averages or published information). The dynamic dose delivery model extracted RT treatment plans, CT planning image sets, dynamic dose distributions, and contoured organ structure sets from DICOM format files in order to determine time-dependent radiation doses for each voxel of each organ of interest. The simulation modeled a pool of CLs, which flowed through each organ and accumulated a distribution of radiation doses. This accumulation was a function of each lymphocytes position in space, dictated by the blood-flow model, and the time-dependent radiation dose from the treatment plan. This was used to determine initial cell kill via a dose-dependent model following published data. Finally, time-dependent lymphocyte regeneration and death was determined using a model fit to patient measurement data, as described below. Each component of the model is described in detail in the following sections.

### 2.2 Blood Flow Stimulation

The pool of CL was simulated using a total blood volume of 5 L, which moves through the patient's body assuming a heart-to-heart circulation time (H2H) of 60 s. (A patient-specific value for blood volume and circulation time can be used as well). This is defined as the time it takes for blood to cycle from the left ventricle, throughout the body (the systemic circulation), then back to the right atrium and right ventricle of the heart, then to the lungs to exchange oxygen and carbon dioxide (the pulmonary circulation), and finally return to the left atrium of the heart. The simulation computes the blood flow through each organ separately, to account for differences in organ size and the rate of blood flow through each organ. For each organ, the blood flow velocity is determined by the Equation 1:

$$v = \frac{5000 ( {cm}^{3} ) \times C O}{H 2 H ( s ) \times [ B V D \times {A}_{CS} ( {cm}^{2} ) ]} ( cm / s ) \qquad ( 1 )$$

Here, CO is the cardiac output percentage, which describes the fraction of total cardiac output flowing to an organ. BVD is the blood volume density, or the volume percentage of an organ that is filled with blood. A_{cs} is the average axial cross-sectional area of the organ, and the product (BVD×A_{cs}) is the amount of blood that is contained in that axial slice of that organ. By using an approach based on the cross-sectional area of each organ, blood flow through complex organ shapes could be modeled that would be accurate on average over the course of a full treatment.

Using this approach, the following regions were simulated: individual great vessels (aorta, vena cava, pulmonary artery), lungs, heart, liver, spleen, and stomach in the case of tumors in the lower thorax. flow through “other organs” was also modeled by subtracting the above regions from the total external contour. Given a set of voxels S inside the patient, and a set O of organs already contoured, the “other organs” were given by Sₒₜₕₑᵣ=S−∪_{i∈O} Sᵢ. For each organ, A_{cs} was computed using the patient's CT images, while CO was obtained from published results. The cardiac output for “other organs” was given by COₒₜₕₑᵣ=1−Σ_{i∈O} COᵢ. For great vessels, lungs, and the heart, CO=100% was used as all blood passes through them with each circulation. This resulted in great vessel average blood velocities of ˜8.7 cm/s for aorta, ˜13.4 cm/s for vena cava, and 7.3 cm/s for pulmonary artery, which are close to published values.

The radiation dose delivery to the CL pool was simulated over time by dividing the total dose from each treatment beam into fractions and subsequently into discrete time-steps. This time interval, dt, was organ dependent and defined by the time required for a circulating cell to traverse an image voxel:

$$d t = \frac{d x}{v} \qquad ( 2 )$$

Here, dx is the voxel length of 2.5 mm and v is the organ-specific blood velocity defined above. The dose from each beam was then divided into N_{seg}=T/dt segments where T is the time required to deliver the entire beam. Each beam segment was further divided into doses delivered to the individual organs listed above. This was done by applying logical masks from the contoured structure sets and enabled the dose to be applied to each structure while avoiding leakage into other organs. For a given organ O, the dose delivered in one beam segment can be computed using the dose D of a single beam for a single treatment fraction and the organ's spatial mask mask_{O}.

$${D}_{O} = \frac{D [ {mask}_{O} ]}{{N}_{s e g}} \qquad ( 3 )$$

The total blood volume was modeled by a blood matrix of size N×1, where

$$N = \frac{5000 {cm}^{3}}{0.25 \times 025 \times 0.25 {cm}^{3}} = 320000$$

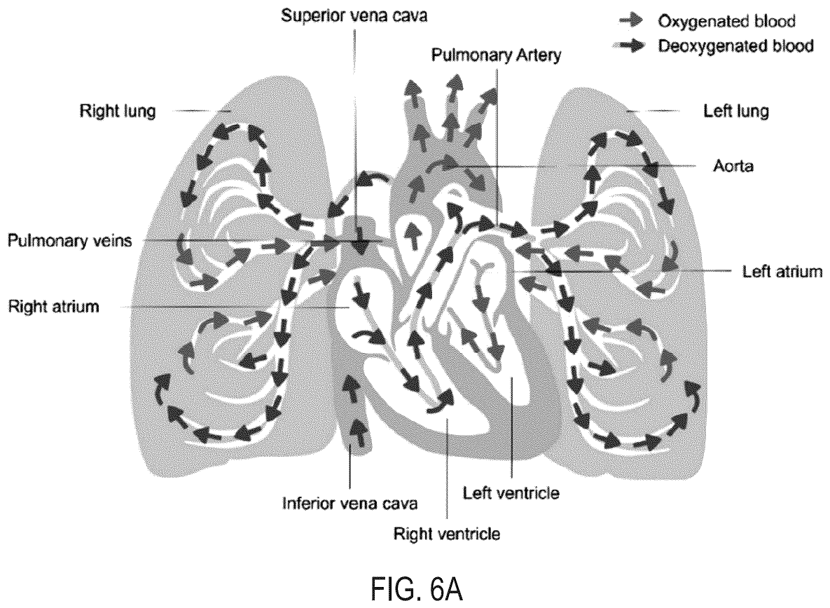

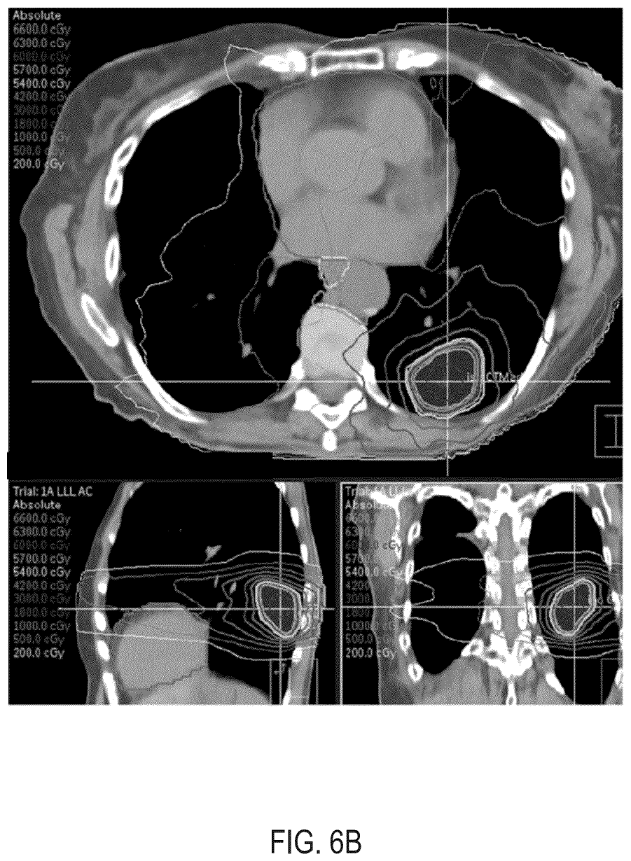

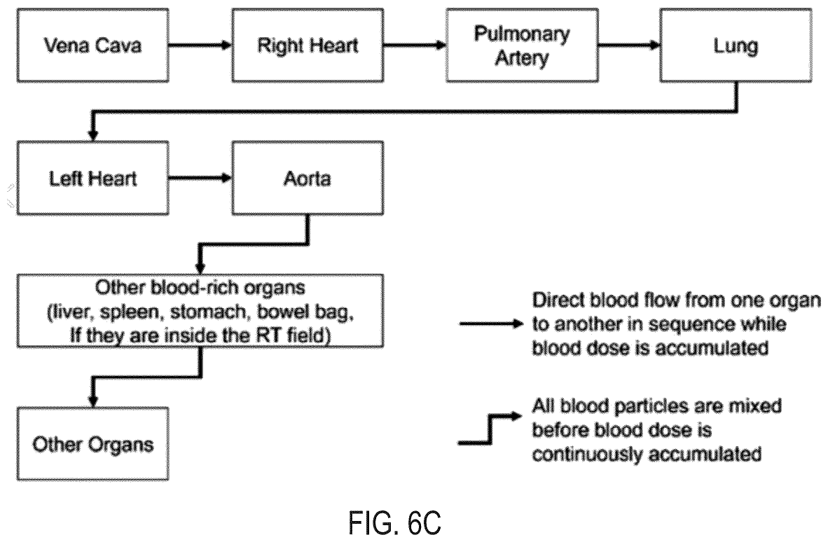

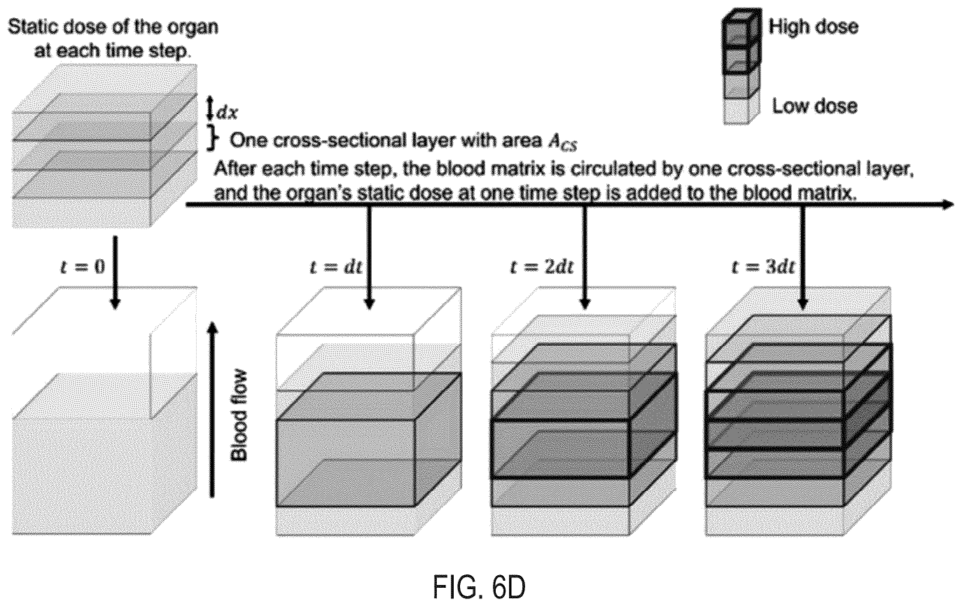

blood particles and 0.25×0.25×0.25 cm³ is the volume of each dose voxel from the treatment planning system dose map. For each organ, the contoured logical mask was applied to the blood matrix to ensure that dose was only applies to the section of blood “inside” the organ at a given time. Every dt seconds, the dose for a given beam segment and organ was applied and the blood matrix was circulated by one organ cross-sectional layer in the superior-inferior direction. During the time required to deliver the entire beam, the blood matrix entries, and the CL they represent, accumulate radiation dose as they move through each organ. This process was repeated for each organ listed above, including the “other organs,” for each beam and each treatment fraction in the patient's plan. Between each beam and treatment fraction, the blood matrix was shuffled to simulate the mixing of blood throughout the body. The result was a blood matrix whose entries captured the dose accumulated by CL in the patient's blood pool over the course of a radiation therapy treatment. FIG. 6C-6D represents the model structure schema and blood matrix dose accumulation due to blood flow through the radiation beam in space and time.

### 2.3 Lymphocyte Toxicity K(t) Simulation

To translate accumulated dose into cell death fractions, a kill function K(Dᵢ) was used where Dᵢ is an entry of the blood matrix representing the dose accumulated by an individual CL after a radiation treatment is administered. A linear-quadratic (LQ) model for lymphocyte kill was used:

$$K ( {D}_{i} ) = 1 - {e}^{- ( α {D}_{i} + β {D}_{i}^{2} )} \qquad ( 4 )$$

Because in vivo lymphocyte radiotoxicity data is not readily available, the parameters α, β were fit to published in-vitro survival curves from Nakamura et al. (α=0.255, β=0.147). This model predicts 15% kill for a dose of 0.5 Gy, 33% kill for a dose of 1 Gy, 67% kill for a dose of 2 Gy, and 88% kill for a dose of 3 Gy. Cumulative dose to CLs has a wide range from 0 to Dₘₐₓ, and the total lymphocyte kill is given as:

$${K}_{0} = {\sum }_{{D}_{i} = 0}^{Dmax} {N}_{i} ( 1 - {e}^{- ( α {D}_{i} + β {D}_{i}^{2} )} ) = {N}_{0} - {\sum }_{{D}_{i} = 0}^{Dmax} {N}_{i} {e}^{- ( α {D}_{i} + β {D}_{i}^{2} )} \qquad ( 5 )$$

Here Nᵢ are the number of cells receiving dose Dᵢ, and N₀=Σ_{D}_{ᵢ}₌₀^{D}^{ₘₐₓ}Nᵢ is the pre-treatment ALC value.

### 2.4 Modeling Time-Dependent ALC

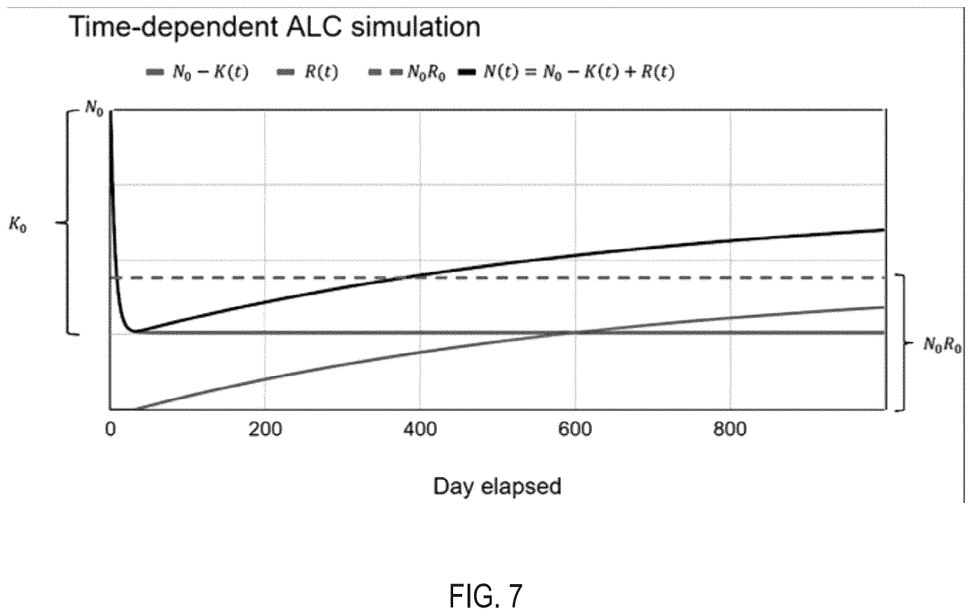

FIG. 7 shows the functional form used to predict ALC over time following treatment. The patient's lymphocyte count as a function of time is given by Equation 6 where K(t) is a time-dependent function for lymphocyte toxicity, and R(t) is a time-dependent function for lymphocyte regeneration.

$$N ( t ) = {N}_{o} - K ( t ) + R ( t ) \qquad ( 6 )$$

$$K ( t ) = {K}_{0} \times ( 1 - {e}^{- at} ) \qquad ( 7 )$$

$$R ( t ) = {N}_{0} \times {R}_{0} \times ( 1 - {e}^{- b ( t - 30 )} ) if t \geq 30 \qquad ( 8 )$$

$$and R ( t ) = 0 if t < 30 ,$$

Cumulative ALC data from patient blood draws was used empirically to model the time-dependent lymphocyte function. It exhibits an exponential decay up to a time of 30 days where a nadir point was observed, and a gradual replenishment afterwards following lung SBRT. Swanson et al. also found a nadir point in absolute lymphocyte reduction around four week time point for different types of cancers treated with standard fractionation RT. The measured time dependence of lymphocyte kill and recovery was modeled using two exponential functions: (i) a time-dependent kill function with Equation 7, where a is an unknown decay constant and t is the number of days elapsed since radiotherapy treatment initiation; and (ii) a time-dependent replenishment rate R(t), which represents the release of new lymphocytes into the bloodstream from primary and secondary lymphoid organs over time, as well as the proliferation of new cells which depends on the pre-treatment immune status of a given patient. It is assumed replenishment occurs following the nadir at 30 days, and model it empirically using Equation 8. Here, Rₒ, b are parameters to be estimated empirically from available longitudinal blood draw data.

Model parameters a, b, and R₀ were optimized to minimize the χ2 difference between simulated lymphocyte survival fractions and measured blood-draw data across the patient cohort using a 10-fold cross-validation process. The cohort was segmented into ten distinct groups, and ten models were fit using 90% of the patients to optimize and evaluated using the remaining 10%. This process allowed us to evaluate model performance on all available data while avoiding overlap between training and test sets. When comparing post-treatment ALC between the output of the model and measurement, the regeneration was projected from the nadir point to the measured ALC date for each patient.

### 2.5 Patient Cohort

Patients who underwent SBRT for NSCLC were included in this institutional review board approved retrospective analysis. Imaging volumes and SBRT treatment plans collected for 64 patients in total were used to optimize and validate the model. Table 1 describes the characteristics of measurement data (patient, dosimetric, and ALC) used for this study, with the blood-rich organ (heart+ great vessels) dose levels that has shown to significantly correlate with post SBRT ALC loss. All plans were optimized to meet the dosimetric criteria defined in the radiation therapy oncology group (RTOG) 0915 (for peripheral tumors, 59.4% of patients) or 0813 (for centrally located tumors, 40.6% of cases) protocols. All plans were designed for delivery on a Varian linear accelerator using either a step-and-shoot intensity-modulated radiation therapy (IMRT) (67.2% of plans), a volumetric-modulated arc therapy (VMAT) delivery (29.7% of plans), or a three-dimensional conventional radiation therapy (3D-CRT) delivery (3.1% of plans).

TABLE 1
Patient and dosimetric characteristics

| Characteristic | | All patients (n = 64) | |
|---|---|---|---|
| Gender | Male | 28 | |
| | Female | 36 | |
| Age | median (range), years | 72.5 | (50-92) |
| Race | Caucasian | 53 | |
| | African American | 7 | |
| | Asian | 1 | |
| | Other | 3 | |
| PTV volume | median (range), cc | 29.34 | (4.76-403.15) |
| RT scheme | 12 Gy × 5Fx (BED^{a} = 132 Gy) | 32 | |
| | 11 Gy × 5Fx (BED = 115.2 Gy) | 1 | |
| | 10 Gy × 5Fx (BED = 100 Gy) | 25 | |
| | 09 Gy × 5Fx (BED = 85.5 Gy) | 3 | |
| | 18 Gy × 3Fx (BED = 151.2 Gy) | 2 | |
| | 17 Gy × 3Fx (BED = 137.7 Gy) | 1 | |
| RT site | Central | 26 | |
| | Peripheral | 38 | |
| Pre-tx ALC | median (range), 10⁹ cells/L | 1.68 | (0.61-3.19) |
| Lung volume | median (range), cc | 3621 | (1649-6433) |
| Lung mean dose | median (range), Gy | 4.20 | (0.88-7.67) |
| Heart volume | median (range), cc | 696 | (310-1624) |
| Heart mean dose | median (range), Gy | 1.45 | (0.02-8.99) |
| Body volume (External) | median (range), cc | 24263 | (12 758-42 913) |
| Body mean dose | median (range), Gy | 1.61 | (0.77-4.24) |
| Heart + GV^{b} mean dose | median (range), Gy | 2.93 | (0.15-8.94) |
| Heart + GV integral^{c} dose | median (range), Gy · cm³ | 2389 | (72-10 291) |
| Heart + GV V5^{d} | median (range), cc | 154.1 | (1.9-671.0) |
| Heart + GV V10 | median (range), cc | 61.3 | (0.0-405.5) |
| Heart + GV V15 | median (range), cc | 19.4 | (0.0-217.3) |
| Heart + GV V20 | median (range), cc | 4.1 | (0.0-105.4) |
| External-PTV mean dose | median (range), Gy | 1.53 | (0.73-3.37) |
| External-PTV integral dose | median (range), Gy · cm³ | 36978 | (14234-86461) |
| External-PTV V1 | median (range), cc | 4613 | (1611-10368) |
| External-PTV V5 | median (range), cc | 2035 | (845-4464) |
| External-PTV V10 | median (range), cc | 1125 | (253-3365) |
| External-PTV V15 | median (range), cc | 601 | (109-2122) |

^{a}BED refers to the biologically effective dose.
^{b}GV refers to the great vessels (aorta, vena cava, and pulmonary artery) combined.
^{c}Integral dose refers to the volume integral of the dose deposited in the patient and is equal to the mean dose times the volume.
^{d}Vx refers to the percentage of the volume that received at least × Gy radiation dose.

### 2.6 Organ-at-Risk Contouring

The following organs-at-risk (OAR) were manually contoured by a set of trained physicians following the RTOG atlas for organs at risk in thoracic radiation therapy, encompassing a region of interest up to a very low threshold dose of 40 cGy per fraction): lungs, great vessels (aorta, vena cava, pulmonary vessels), heart, liver, spleen, and stomach (only for lower thorax tumors). All remaining tissue in the body was combined to a region denoted “Other Organs.” Example masks showing this discretization are shown in FIG. 1B.

### 2.7 Lymphocyte Measurements

ALC measurements were recorded pre-treatment and at multiple post-treatment time points. Post-treatment lymphocyte count drops were determined by computing the difference between the first post-treatment ALC and the pre-treatment baseline ALC. Having post-treatment blood data at different time points helps define the time dependence of the predictive model.

### 2.8 Model Accuracy

Model accuracy was assessed by comparing predicted post-treatment ALC with those measured in the post-treatment blood draws. Accuracy was assessed for each patient in the test sets created during the survival function cross-validation process. Differences in prediction accuracy in the presence of specific plan characteristics (tumor location, tumor volume, radiation delivery method, and treatment time) were evaluated to identify systematic errors.

The model was also assessed in terms of its rate of immune suppression prediction accuracy using the cumulative distribution function (CDF). CDF is defined by the probability that the prediction accuracy will have a value less than or equal to a given difference of measurement to prediction.

### 2.9 Dose-Level Contributions to Lymphocyte Kill

To identify the dosimetric drivers of lymphocyte toxicity, contributions were compared to cell kill from different blood dose levels. This is important to assess whether RIIS is driven primarily by focused high-dose regions or distributed low doses. The kill contribution C (D) of dose D for a given patient is given by Equation 9:

$$C ( D ) = \frac{K ( D ) \times P ( D )}{{\sum }_{d} K ( d ) P ( d )} \qquad ( 9 )$$

where K(D) is the lymphocyte kill function value at dose D and P(D) is the percentage of blood cells receiving dose D. D was binned with a resolution of 0.1 Gy.

### 2.10 Dependence of Treatment/Patient Characteristics on Immune Suppression Model

To validate the model's ability to correlate the post-treatment RIIS with key patient/plan characteristics that is observed in the measurement, the Spearman correlation functions were evaluated between predicted post-treatment ALC and five variables: pre-treatment ALC, minimum distance between PTV and heart, days elapsed from treatment initiation to post-treatment ALC measurement, PTV volume, and treatment delivery time. Correlations between patient measurements and each variable were also evaluated and compared them to the model predictions. To evaluate systematic differences between patient/plan characteristics, differences in cell kill contribution were compared between the above five parameters as well as age of the patient, treatment modality (step-and shoot IMRT/3D/arc deliveries), and tumor location (central/peripheral).

### 3. Results

For a typical lung SBRT plan, 320 000 blood particles were simulated and propagated through and out of the dose volume. Typical times to compute post-treatment ALC were 12-field 3D plan (23 s), 11-field IMRT plan (37 s), and 2-arc VMAT plan (37 s). These short simulation times indicate that the model could interface with the treatment planning system and provide near-real time feedback to optimize plans with immune suppression considerations.

### 3.1 Accumulated Blood Dose Distribution

The model predicts a distribution of radiation doses accumulated by the patient's blood pool. FIG. 3a top shows the average distribution for all patients after receiving a single treatment fraction. The mean accumulation is low (0.25 Gy), although it increases after all five treatment fractions have been administered (1.15 Gy, see FIG. 3a middle). These plots combine the individual dose distributions for each patient in the cohort, which are narrowly distributed around different mean values.

The location and width of the blood dose distribution are determined by several factors. Patients with larger PTVs accumulate higher blood dose levels (example in FIG. 3b top four PTV volumes: <20 cc, between 20 and 40 cc, between 40 and 60 cc and >60 cc), due to larger treatment fields, leading to larger volumes of radiation dose exposures. The location of the tumor in the lung also has an effect, with central tumors resulting in higher dose accumulation than peripheral tumors (FIG. 3b middle). This occurs due to the closer proximity to great vessels and heart, which have a higher blood density than surrounding tissue in the case of central tumors. For all subgroups of the patient cohort, the prediction fraction of blood receiving greater than 3 Gy (corresponding to the highest kill fraction reported in Nakamura et al.) was negligible.

### 3.2 Dose-Level Contributions to Lymphocyte Kill

The LQ model (Equation 4) converts the accumulated dose distribution into an initial lymphocyte kill fraction. Using the kill contribution metric (Equation 9), the delivered dose regions were evaluated which led to the highest lymphocyte reduction. In most cases, the highest percentage of lymphocyte toxicity for lymphocytes in the circulating blood pool came from doses around 1.5 Gy. As FIG. 3a bottom shows, for all patients combined, lymphocyte toxicity was dominated by low dose levels (33.7% of toxicity came from doses<1 Gy, 75.3% of toxicity came from dose<1.5 Gy) despite the lower cell kill probabilities at these levels. Only 8.2% of the lymphocyte toxicity came from cells absorbing more than 2 Gy.

### 3.3 Predicted Absolute Lymphocyte Counts

Using the time-dependent ALC model (see Methods), each patient's ALC was forecasted at their respective post-treatment measurement days. The model was fit and tested using a ten-fold cross validation procedure. The model parameters for (a,b, and R₀) and accuracy for each iteration are given in Table 2. The histogram for the raw absolute ALC difference (prediction-measurement) is shown in FIG. 4, with the color indicating which fold one particular ALC difference was on.

TABLE 2
Results of optimized parameters and prediction accuracy for
the training and testing set for each of the 10 folds.

| | | | | | | ALC Difference between prediction | | | |
|---|---|---|---|---|---|---|---|---|---|
| | | | | | | and measurement (10⁹ cell/L) | | | |
| | N | N | a | | b | Mean- | SD- | Mean- | SD- |
| Fold | train | test | (day⁻¹) | R₀ | (day⁻¹) | training | training | testing | testing |
| 1 | 57 | 7 | 0.5261 | 0.7020 | 0.0009 | 0.21 | 0.15 | 0.35 | 0.15 |
| 2 | 57 | 7 | 0.5261 | 0.7020 | 0.0009 | 0.23 | 0.31 | 0.20 | 0.31 |
| 3 | 57 | 7 | 0.5261 | 0.7022 | 0.0014 | 0.22 | 0.27 | 0.33 | 0.27 |
| 4 | 57 | 7 | 0.5261 | 0.7020 | 0.0009 | 0.22 | 0.12 | 0.26 | 0.12 |
| 5 | 58 | 6 | 0.5276 | 0.7295 | 0.0009 | 0.23 | 0.10 | 0.26 | 0.10 |
| 6 | 58 | 6 | 0.5263 | 0.8009 | 0.0012 | 0.24 | 0.10 | 0.14 | 0.10 |
| 7 | 58 | 6 | 0.5261 | 0.7049 | 0.0008 | 0.23 | 0.19 | 0.19 | 0.19 |
| 8 | 58 | 6 | 0.5262 | 0.7054 | 0.0009 | 0.23 | 0.09 | 0.18 | 0.09 |
| 9 | 58 | 6 | 2.9997 | 0.8040 | 0.0007 | 0.22 | 0.24 | 0.30 | 0.24 |
| 10 | 58 | 6 | 0.5261 | 0.7020 | 0.0014 | 0.24 | 0.17 | 0.18 | 0.17 |

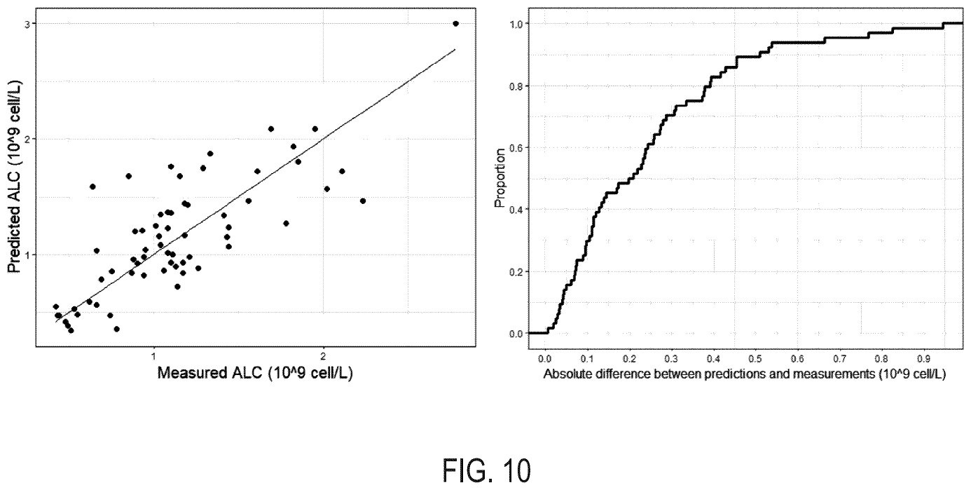

FIG. 10 compares post-treatment ALC model predictions to patient measurements. The left panel plots the predicted post-treatment ALC against the measured ALC count. The right panel shows the CDF (CDF of patient predictions achieving a given accuracy. Predictions differed from measured values by an average (SD): 0.24 (0.21)×10⁹ cell/L (or 13.7% (10.3%) compared to pre-treatment ALC). Eighty-nine percent of the patients have a difference between prediction and measurement smaller than 0.5×10⁹ cells/L. The measured pre-treatment ALC values have a mean (range) of 1.68 (0.61-3.19). The model's mean error rate is lower than half of the lowest ALC value, indicating it has sufficient sensitivity to detect whether a patient is expected to develop treatment-related Grade 3 lymphopenia (post-treatment ALC<0.5 lymphopenia per NCI guidelines). The model is able to predict post-treatment ALC<0.8 (grade 3 lymphopenia), with a sensitivity of 81%; and a specificity of 98% (due to the low statistic, G2 lymphopenia were tested as opposed to G3 lymphopenia). The mean (STD) for the difference between prediction and measurement over the 10 folds for different sub-groups (pre-treatment ALC, age, treatment modality, post-treatment blood draw time point, tumor location, treatment delivery time, and tumor volume) are shown in Table 3.

TABLE 3
A summary of the mean (STD) for the difference between
prediction and measurement over the 10 folds of the
predictive model within each subset of variables.

| | Absolute | Percent | | |
|---|---|---|---|---|
| | difference | difference | | |
| | (10⁹ cells/L) | (%) | | Count |
| Pre Tx ALC (10⁹ cells/L) | | | | |
| <1.0 | 0.08 (0.08) | 9.4 | (9.9) | 8 |
| 1.0-1.5 | 0.18 (0.12) | 14.1 | (8.3) | 16 |
| 1.5-2.0 | 0.24 (0.22) | 14.0 | (11.7) | 20 |
| ≥2.0 | 0.35 (0.23) | 15.0 | (10.6) | 20 |
| Age | | | | |
| <67 | 0.27 (0.23) | 14.8 | (12.0) | 16 |
| 67-73 | 0.23 (0.14) | 14.4 | (8.2) | 16 |
| 73-80 | 0.25 (0.27) | 14.7 | (13.4) | 14 |
| ≥80 | 0.22 (0.18) | 11.4 | (7.6) | 18 |
| Modality | | | | |
| 3D-CRT | 0.15 (0.08) | 10.0 | (1.7) | 2 |
| IMRT | 0.26 (0.20) | 14.8 | (10.0) | 43 |
| Arc | 0.20 (0.22) | 11.7 | (11.2) | 19 |
| Measurement day | | | | |
| <30 | 0.18 (0.13) | 11.7 | (8.0) | 19 |
| 30-180 | 0.26 (0.24) | 14.5 | (11.8) | 35 |
| ≥180 | 0.29 (0.21) | 15.0 | (8.2) | 10 |
| Tumor location | | | | |
| Central | 0.19 (0.14) | 11.9 | (7.7) | 26 |
| Peripheral | 0.27 (0.23) | 15.0 | (11.6) | 38 |
| Treatment time (s) | | | | |
| <200 | 0.21(0.24) | 11.8 | (12.1) | 16 |
| 200-300 | 0.23 (0.20) | 13.5 | (8.8) | 23 |
| ≥300 | 0.27 (0.20) | 15.2 | (10.5) | 25 |
| Tumor volume (cc) | | | | |
| <20 | 0.30 (0.26) | 16.3 | (12.9) | 15 |
| 20-40 | 0.21 (0.19) | 11.8 | (8.8) | 23 |
| 40-60 | 0.29 (0.22) | 15.3 | (10.0) | 11 |
| ≥60 | 0.20 (0.14) | 12.9 | (9.9) | 15 |

Variables considered are Pre-treatment ALC count, age, treatment modality, day of post-treatment blood draw, central vs peripheral, treatment delivery time, and PTV volume size.

### 3.4 Prediction Accuracy Dependence on Patient and Delivery Characteristics

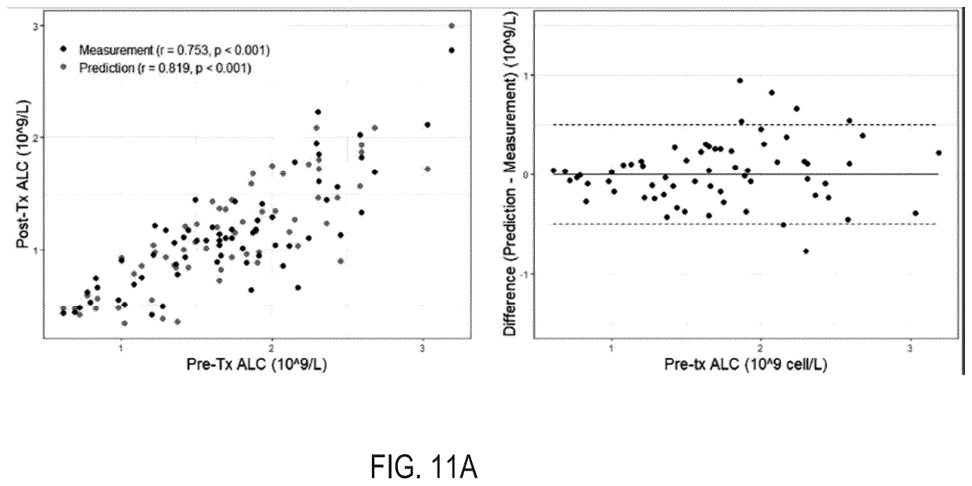

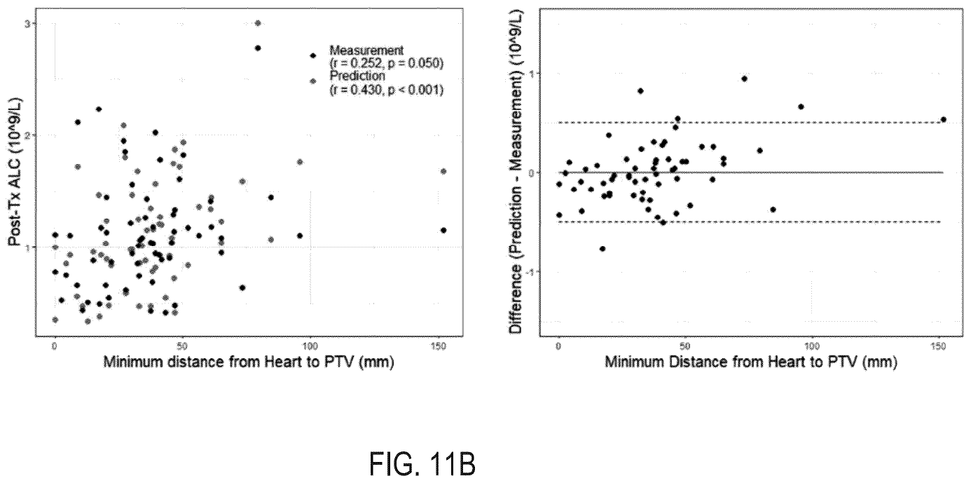

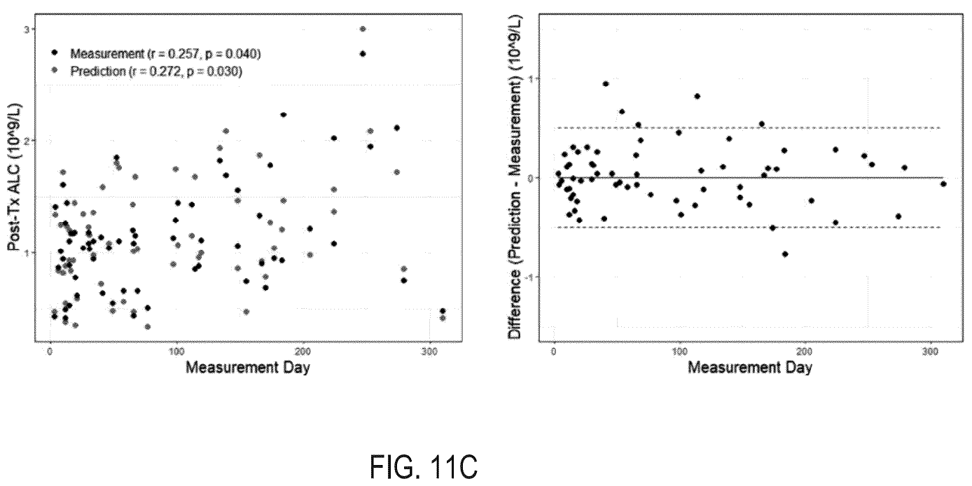

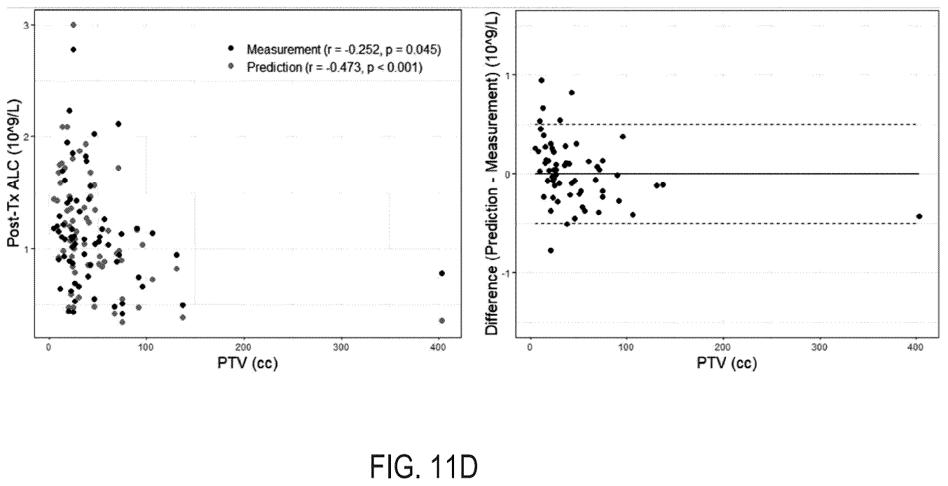

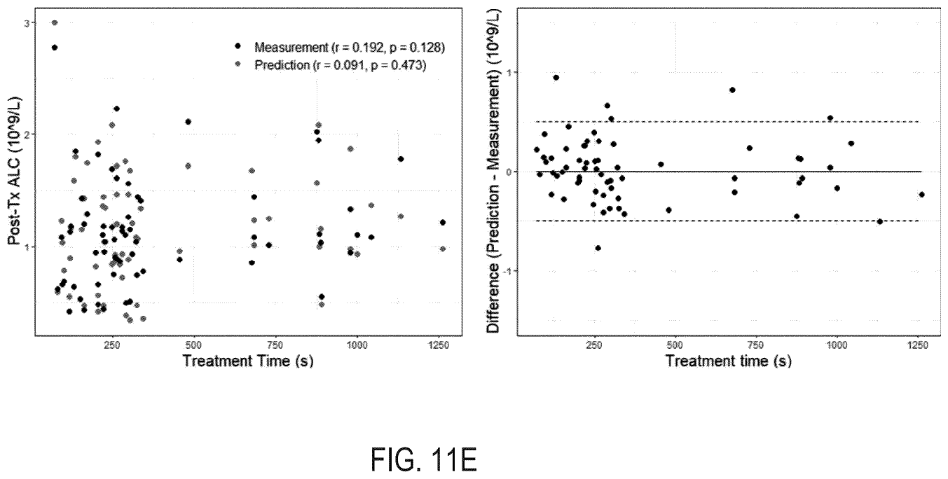

Different key treatment plan/patient characteristics can affect RIIS differently. To validate the model further, it was next evaluated whether the model predictions recreate observed relationships between plan characteristics and RIIS. FIGS. 11A-11E shows post-treatment ALC as a function of five variables. The left column shows the post-treatment ALC as a function of the plan characteristics for both measurement and simulation and the right column plots the model accuracy (absolute difference between prediction and measurement). Variables shown are (A) pre-treatment ALC, (B) minimum distance between PTV and heart, (C) days elapsed from treatment initiation to post-treatment ALC measurement, (D) PTV volume, and (E) treatment delivery time. A trend was not observed between any of these plan characteristics and predictive accuracy. Some notable observations from these comparisons were:

### 3.4.1 Pre-Treatment ALC

As the pre-treatment ALC increases, post-treatment ALC values also increase significantly for both the measurement [r=0.75 (p<0.001)] and the prediction [r=0.82 (p<0.001)].

### 3.4.2 Minimum Distance Between PTV and Heart

Patients whose PTV is further from the heart accumulate lower RT dose in the blood-rich organs, leading to higher post-treatment ALCs [r=0.25 (p=0.05)]. The model correctly predicts a positive correlation between the minimum PTV-heart distance and post-treatment ALC [r=0.43 (p<0.001)].

3.4.3 Days Elapsed from Treatment Initiation to Post ALC Measurement

Regeneration (Equation 7) leads to higher measured ALCs [r=0.26 (p=0.04)] at later measurement days. The model predictions capture this positive correlation [r=0.27 (p=0.03)].

### 3.4.4 PTV Volume

Patients with larger PTV receive larger volumes of all doses, leading to higher dose accumulation and lower post-treatment ALC [r=−0.25 (p=0.04)]. The model predictions capture this negative correlation [r=−0.47 (p<0.001)].

### 3.4.5 Treatment Delivery Time

There is no observed trend with post-treatment ALC in measurement [r=0.19 (p=0.13)] or prediction [r=0.09 (p=0.47)] with treatment delivery times varying from 99 to 683 s at these low number of treatment fractions.

### 4. Discussion

As described herein, a model was presented which predicts radiation-induced lymphocyte reduction following lung SBRT by simulating the dose delivered to CLs in the bloodstream. This approach is patient-specific, using three-dimensional dose maps from treatment planning software, varied treatment times, and organ-specific blood flows. The model predicts initial lymphocyte death using a linear-quadratic survival curve and predicts time-dependent lymphocyte replenishment based on patients' baseline immune status. Thus, in some embodiments, an immune response predictor may utilize as an input a baseline lymphocyte count, specific measured T cell subtypes, and similar measurements or test results pertaining to the patient's immune system status and patient-specific immune factors prior to commencement of RT. (In further embodiments, the same or a similar model can be provided with updated imaging and immune information in the event an immunogenic effect is sufficient to warrant exploring possible reduction of future dosing/regimens.) The model could accurately predict post-treatment ALC on an independent test patient dataset, with an average absolute error of 0.24±0.21×109 cells/L and 89% of patients having an error below 0.5×109 cells/L. The model also predicts relationships between patient/treatment characteristics and measured lymphocyte depletion that are consistent with previous observations. This includes positive correlations between post-treatment ALC and measurement day as a result of lymphocyte replenishment following a nadir point, and negative correlations between post-treatment ALC and target size, as well as heart proximity to PTV. A strength of this blood circulation model is that it was developed and validated using patient-specific data to predict immune suppression.

Existing modeling approaches for RIIS belong to three categories: dose volume-based calculations, blood flow-related simulations with or without surrounding organs of the PTV, or deep learning-based models. One notable blood flow model is Beekman et al.'s stochastic model that simulates systemic blood flow in the human body based on a previously designed compartmental model applicable to any anatomical site. This model is more explicitly grounded in flow physics than the approach described herein, and also requires registration of each patient's anatomy with a phantom vasculature to predict blood dose. The latter step requires great precision when registering the thorax anatomy (which encompasses a large volume of low-dose regions) with smaller critical organs such as the aorta, especially as blood flow rates vary so much between great vessels and the rest of the body. This increased complexity allowed them to study effects such as patient breathing which the model herein neglects, but it comes at the cost of significantly longer runtimes (Beckman: 64 s to model blood flow and 61 s to accumulate dose on an MI iMac with a time step of 0.05 s. Ours: 38 s end-to-end on an Intel Xeon E5-2630 Processor with a time step of ˜ 0.03 s for the blood-rich organs). This stochastic approach to model flow through lower-dose organs in the thorax is aimed to be accurate on average over the course of a treatment. Indeed, Beckman et al. found that changes to blood dose due to breathing tended to average out and be negligible. The model described herein also avoids concerns with registering a phantom volume by using each patient's specific anatomy. Finally, the model uses the blood dose distribution to predict a post-treatment ALC, which accounts for time-dependent lymphocyte death and regeneration. By linking blood dose to cell kill and validating the predictions for the first time, using SBRT patient data for absolute lymphocyte reduction, the applicability of the model for mitigating RIIS was demonstrated.

Simpler models that use mean organ doses and subsets of organs in the thorax to compute immune suppression may have even shorter evaluation times, but will have less predictive power according to Beckman et al. Other recent models have examined ALC reduction in RT treatments with standard fractionation. Jin et al. specifically examined RT for pancreatic and used DVH information to make their predictions, rather than the three-dimensional dose maps that informed the dynamic dose delivery simulation. Ebrahami et al. developed a hybrid deep learning model for esophageal cancer. Both models have limited use for SBRT, where the time-dependent immune suppression is not smoothly varying as in standard fractionation.

In addition to predicting lymphocyte depletion from Lung SBRT, this model also provides tools to potentially mitigate its immunosuppressive impact without altering efficacy. With near-real time predictive abilities, this model could interface with treatment planning systems, to predict lymphocyte depletions for prospective plans, these could be evaluated alongside traditional organ-at-risk dose metrics in determining a plan's quality. Furthermore, the analysis of dose-dependent lymphocyte kill contributions can link specific treatment plan characteristics with lymphocyte depletion and help identify general techniques that may reduce it. After creating an initial treatment plan, one can estimate the total lymphocyte toxicity stemming from specific blood-dose levels (i.e., whether the majority of lymphotoxicity from the plan is due to a large volume of blood receiving a small dose or a smaller volume receiving a higher dose). This information can then be used to prioritize physical dose levels (i.e., standard RT planned doses) to minimize overall cell kill. This method is being evaluated prospectively in an NCI-funded clinical trial (NCT04273893). One potential application of this model will be to optimize fractionation strategies from an immune perspective. Accounting for time-dependent lymphocyte reduction following RT may also help elucidate optimal timing for combined radiation and immunotherapy.

The results achieved in the experimental study can be leveraged in several ways to reduce lymphotoxicity. For example, it was found that patients with centrally located tumors experience a similar degree of lymphocyte depletion as patients with peripheral tumors despite being treated with 20% lower prescription doses (50 vs. 60 Gy). This finding, while to some extent counterintuitive when considering spatial locations and the patient measurements, likely stems from differences in specifics of the dose distributions in these cases. Thus, the inventors determined that non-optimized treatment plans for centrally located tumors deliver larger doses to blood-rich organs in the thorax (i.e., the great vessels and heart) resulting in more CLs accumulating a toxic dose per unit dose to the tumor. Developing optimized treatment plans for centrally located tumors that explicitly avoid blood-rich areas, and areas exhibiting large degrees of lymphatic cell circulation, may bring these situations closer to parity.

The experiments performed validated the techniques and approaches described herein, by deploying a predictive model that accurately predicts lymphocyte depletion following Lung SBRT as well as the onset of lymphopenia in a cohort of 64 patients under ten-fold cross validation. This model with ˜38-s end-to-end prediction time has the capability to be interfaced with treatment planning systems to prospectively reduce immune cell toxicity without compromising treatment efficacy during treatment planning. The proposed RIIS prediction method is adaptable to predict RIIS in other dose fractionations in the lung as well as other disease sites.

However, as noted above, the scope of this disclosure is not limited to merely predicting immune response, nor to SBRT, nor to lung cancer or thoracic RT. Instead, the predictive model (and similar models) can be leveraged in processes for generating, optimizing, evaluating, and selecting patient-specific, disease-specific RT plans that balance a variety of goals that can be informed by the output of such prediction model (e.g., the methods of FIGS. 4-5).

Thus, in some embodiments, dose constraints may be established for a variety of tissues within the region of interest around one or more tumors/lesions/other cancer areas in a patient, based on imaging data. Based on imaging and modeling of the tumor and surrounding tissues, dose constraints are set for each region. Cancer tissue can be assigned higher dose maximum thresholds and/or dose minimum thresholds to ensure effective radiation, while immune-sensitive structures (e.g., lymphatic structures, lymphoid organs, lymphoid tissues, bone marrow, tumor-draining lymph nodes, blood-rich organs) are assigned lower dose constraints, such as maximum dose thresholds. Immune-sensitive structures like lymph nodes, the spleen, and parts of the bone marrow are specifically labeled to receive as little radiation as possible to protect circulating immune cells, particularly lymphocytes. In addition, or separately, other sensitive organs and structures can be assigned dose thresholds lower than the cancer tissue. These thresholds may be entirely user defined, entirely predefined according to software settings based on population averages, or default settings adjustable by a user or according to desired user goals for immune response.

Via an iterative process, RT plans can be generated that fit within these thresholds and constraints. As described above, modified MCO and KBP techniques that account for immune sensitive structures can be employed. An optimization/minimization function can be used to attempt to keep radiation dose to immune sensitive structures at a minimum while still achieving the other dose constraints and objectives. These techniques and algorithms iteratively adjust dose distributions, creating a spectrum of solutions that balance high-dose delivery to the tumor with dose sparing to OARs and radiation-sensitive immune structures. In some embodiments, optimal tradeoffs are presented in a set of candidate plans. These algorithms (e.g., KBP) may also offer dose distribution suggestions and “learned” dose constraints for OARs and sensitive immune structures, as an aid to the iterative process to approach candidate plans having a higher likelihood of success.

Next, the candidate plans that are generated and optimized per the above are processed by a software-based, automatic immune response predictor model. In some examples, the immune response predictor may predict immune suppression by using an optimization algorithm that incorporates a dynamic model of blood and lymphatic flow in the specific patient's region of interest. This model simulates how blood circulates through irradiated areas over time, estimating the dose accumulation in immune cells. By using patient-specific parameters to inform circulation dynamics, the predictor can be used to cull or refine candidate plans to avoid excessive dose exposure to blood-rich regions, enhancing the preservation of circulating lymphocytes. Thus, in addition to simply utilizing dose constraints for static regions, the predictor gives dynamic information about impact of a radiation dosing so as to inform the optimization algorithm in regard to moving cells.

Optionally, the candidate plans that meet the dose constraints and have suitable impact on circulating cells can be further analyzed to determine optimal dose distributions. For example, systems and methods may use dose-volume histograms (DVHs) to quantify the dose distribution of a given optimal plan across target tumors, OARs, and immune tissues. For immune preservation, the DVH provides an easy way for planners to examine dose levels, and ensure low-dose goals are achieved, for lymphatic structures and blood-rich organs, minimizing the dose to structures critical for immune function.

Some systems and methods may include automated plan refinement, where an optimization algorithm makes iterative adjustments to one or more candidate optimal plans, to improve immune sparing or promote generation of tumor-directed immune cells. Thus, the system can fine tune overall RT plans and radiation beam parameters, including DICOM RT files, to set RT treatment parameters such as collimator positioning, gantry angles, and dose rates to further optimize treatment toward the goals described above.

After generating and refining plans, the predictive model may be used to generate information for a physician or other user to help evaluate one or more optimal plans' impact on immune response, such as radiation-induced immune suppression (RIIS) and promotion of beneficial immune cells. Plans are assessed based on predicted lymphocyte depletion and potential immune-modulatory effects, and a physician may selecting a plan via a user interface that best meets both therapeutic and immune-preserving goals or select a feasible balance of these goals (and the system chooses the associated optimal plan).

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims.

The phrase “such as” should be interpreted as “for example, including.” Moreover, the use of any and all exemplary language, including but not limited to “such as”, is intended merely to better illuminate associated disclosure and does not pose a limitation on the scope of the disclosure unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

1. A method of generating a treatment plan based on predicting an immune effect induced by ionizing radiation in a subject, the method comprising:
assembling input data, wherein the input data includes imaging data of a subject, information relating to blood circulation of the subject, information relating to a radiation-based cancer therapy of the subject, and information concerning desirability of the immune effect;
delivering the input data to a system for predicting immune response following radiation therapy (RT), wherein the system models immune cell toxicity in circulating blood caused by the RT;
receiving an output from the system, comprising a predicted immune effect;
determining a treatment plan for the subject using the output; and
generating a report including at least one of the output or treatment plan.

2. The method of claim 1, wherein the input data includes computed tomography (CT) data, RT structure sets, treatment plan parameters, and dose maps.

3. The method of claim 2, wherein the treatment plan parameters include at least one of radiation dosage, duration, and frequency.

4. The method of claim 2, wherein the output from the system accounts for time-dependent lymphocyte death and regeneration, to provide an indication of changing immune effect for at least a set of times after an RT treatment.

5. The method of claim 1, wherein the system for predicting immune response includes a linear-quadratic (LQ) model.

6. The method of claim 5, wherein the output of the system predicts radiation-induced immune suppression (RIIS), including a change in a count of at least one blood cell type and at least one lymphocyte sub-population.

7. The method of claim 6, wherein the at least one lymphocyte sub-population includes T cells, B cells, or natural killer (NK) cells.

8. The method of claim 7, wherein the T cells include CD3+. CD4+, CD8+, CD19+, or CD56+.

9. The method of claim 5, wherein the LQ model models at least one lymphocyte sub-population kill as: K(Dᵢ)=1−e^{-(αD}^{ᵢ}^{+βD}^{ᵢ}^{²}⁾, where Dᵢ is an entry of a blood matrix representing a dose accumulated by an individual circulation lymphocyte after RT.

10. The method of claim 5, wherein the LQ model models interaction between (1) a time-dependent RT delivery, (2) a movement of blood, lymphatics, and lymphocytes, and (3) osmosis between primary organs, secondary organs, and non-lymphoid organs.

11. The method of claim 10, wherein the interaction further includes (4a) a blood cell kill, (4b) a bone marrow kill and a bone marrow recovery time, or (4c) a combination thereof.

12. The method of claim 1, wherein the RT includes lung Stereotactic Body Radiation Therapy (SBRT).

13. A system for predicting an immune modulation effect induced by ionizing radiation in a cancer patient, the system comprising:
a user interface;
a processor; and
a memory having stored thereon an immune response predictor, and software which, when executed by the processor, causes the system to:
receive imaging data of a region of interest of the cancer patient;
develop a spatial model of the region of interest;
update the spatial model to account for lymphatic and blood flow dynamics of the cancer patient;
determine target structures and avoidance structures within the spatial model;
generate a set of potential RT treatment plans;
evaluate the potential RT treatment plans using the updated spatial model and the immune response predictor, to determine predicted immune responses for the potential RT treatment plans;
select one or more optimal plans based on the predicted immune responses; and
output the one or more optimal plans to a user with information concerning likely immune response for the one or more optimal plans.

14. The system of claim 13, wherein the lymphatic and blood flow dynamics model blood flow within the patient relevant to lymphatic circulation, based on at least one of a blood volume and a blood flow rate of the cancer patient.

15. The system of claim 13, wherein the target structures include at least one tumor target, and the avoidance structures include at least one organ at risk and at least one lymphoid tissue.

16. The system of claim 15, wherein the software further causes the system to receive, via the user interface, an indication from a user determinative of which structures in the imaging data of the region of interest are target structures and which are avoidance structures.

17. The system of claim 16, wherein the software further causes the system to receive, via the user interface, an indication from a user determinative of which avoidance structures are organs at risk and which are lymphoid tissue, and an indication of relative importance of avoidance of such structures.

18. The system of claim 13, wherein the immune response predictor comprises a software module that tracks expected radiation exposure of immune cells as they are simulated to circulate through the region of interest during each RT treatment of a given RT plan, based on the updated spatial model.

19. The system of claim 18, wherein the software further causes the system to provide patient-specific immune factors to the immune response predictor, including a measured baseline lymphocyte count and one or more detected T cell subtypes.

20. The system of claim 18 wherein the immune response predictor determines dose to lymphatic tissues and dose to tumor regions for a given RT plan, and using such determinations predicts a modulation of cytotoxic T cells.

21. The system of claim 18, wherein immune response predictor models lymphocyte kill as K(Dᵢ)=1−e^{-(αD}^{ᵢ}^{+βD}^{ᵢ}^{²}⁾, where Dᵢ is an entry of a blood matrix representing a dose accumulated by an individual circulation lymphocyte after RT.

22. The system of claim 13, wherein the software further causes the system to assess the predicted immune responses for at least a portion of the potential RT treatment plans against user input including a desired immune modulation effect and at least one dose constraint, and present to the user the potential RT treatment plans having predicted immune responses closest to the desired immune modulation effect while meeting the at least one dose threshold.

23. The system of claim 13, wherein the software further causes the system to output information concerning likely change in immune response over time for the one or more optimal plans, and a recommendation for timing of an immunotherapy based on the change in immune response over time.
